(19) United States
(12) Patent Application Publication
(10) Pub. No.: US 2019/0170735 A1
(43) Pub. Date: Jun. 6, 2019

(54) IMMUNO CHROMATOGRAPHY METHOD WITH CENTRIFUGE ISOLATION

(71) Applicant: Marvin LIU, Changzhou (CN)

(72) Inventor: Marvin LIU, Changzhou (CN)

(21) Appl. No.: 16/206,726

(22) Filed: Nov. 30, 2018

Related U.S. Application Data
(63) Continuation of application No. PCT/CN2017/086044, filed on May 26, 2017.

(30) Foreign Application Priority Data
May 31, 2016 (CN) 201610373048.6
Jun. 27, 2016 (CN) 201610477903.8
Jul. 16, 2016 (CN) 201610561226.8
Aug. 3, 2016 (CN) 201610625484.8

(51) Int. Cl. G01N 33/538; B01L 3/00; G01N 33/53; G01N 33/539; G01N 33/543; G01N 21/05

(52) U.S. Cl. CPC G01N 33/538; B01L 3/5023; G01N 33/5302; B01L 2400/0409; G01N 33/5434; G01N 21/05; G01N 33/539

(57) ABSTRACT

Disclosed is an immune chromatography method with centrifuge isolation. A microparticle (colloidal gold and fluorescent microsphere) as a carrier carries an analyte and an intermediate thereof, and a chromatography flow of the microparticle is performed on a solid phase membrane to complete a reaction, thereby improving a capacity of the solid phase membrane to capture and bind the analyte and the intermediate thereof. A centrifugation device is provided to drive a liquid phase to flow on the solid phase membrane for chromatography, thereby effectively reducing non-specific binding between specifically captured chemiluminescent substances and the solid phase membrane and background noise interference from the solid phase membrane, and promoting detection sensitivity.

### CROSS-REFERENCE TO RELATED APPLICATIONS

This application is a continuation of International Application No. PCT/CN2017/086044, filed on May 26, 2017, which claims the benefit of priority from Chinese Application No. 201610373048.6, filed on May 31, 2016, Chinese Application No. 201610477903.8, filed on Jun. 27, 2016, Chinese Application No. 201610561226.8, filed on Jul. 16, 2016 and Chinese Application No. 201610625484.8, filed on Aug. 3, 2016. The contents of the aforementioned applications, including any intervening amendments thereto, are incorporated herein by reference.

### TECHNICAL FIELD

The present application relates to immunoassay technology, and particularly to an immune chromatography method with centrifuge isolation.

### BACKGROUND

Immunoassay technologies using immunology principle have been developed to determine antigens, antibodies, immune cells and chemical components, etc., so that it has a wide application not only in disease diagnosis and health testing for human and animals, but also in sample analysis related to environment, pharmaceutical, food and industry. Currently, the commonly used immunoassay technologies include immunoturbidimetric assay, solid phase enzyme immunoassay, chemiluminescence detection, immunofluorescence labeling, flow cytometry and colloidal gold technology.

Immunoturbidimetric assay, also referred to as immunoturbidimetricassay, can be applied to a quantitative determination through detecting a transmitted light scattered light as a calculation member formed by a light refraction or absorption caused by a complex of a certain size produced with a specific binding of a soluble antigen and antibody in the liquid phase. However, this assay has a limitation in trace detection due to the low sensitivity.

Solid phase enzyme immunoassay technology is carried out using immobilization and enzyme labeling of antigen or antibody. In this assay, the antigen or antibody bound to the surface of the solid phase carrier still has the immunological activity, and the enzyme conjugate of the antigen or antibody not only has the immunological activity, but also possesses the enzyme activity. In the detection, the to-be-tested sample (for determining the antigen or antibody thereof) and the enzyme-labeled antigen or antibody are reacted with the antigen or antibody on the surface of the solid phase carrier in different procedures. This assay has advantages of a high sensitivity, a wide linear response range and an easy automation. However, a long reaction time of the detection limits the application of this assay.

Immunochemiluminescence detection technology can be used to analyze the target at a micro or trace amount due to the high sensitivity, moreover, this technology also has the advantages of an easy operation, a wide linear response range and an easy automation. Therefore, this technology is widely used for sample analysis related to environment, pharmaceutical, food and industry. This technology is also performed using the solid-phase separation and luminescent reagent-labeling of the antigen or antibody, but a long reaction time of the detection and a high requirement for the detection device limit its application.

Though immunofluorescence labeling technology, flow cytometry and colloidal gold technology are also widely used, they all have their respective defects together with common defects of a long reaction time of detection, a low sensitivity or a lack of accuracy.

High sensitivity, rapidity, miniaturization, full quantification, and automation are the development trends of clinical immunoassay technology products, but the existing related products cannot simultaneously achieve such characteristics.

### SUMMARY

The object of the present application is to provide various immune chromatography methods with centrifuge isolation with high sensitivity, short detection time, wide detection linear range and easy operation, including a paper chromatography chemiluminescence assay, a fluorescence immune chromatography assay with centrifuge isolation, and a immune chromatography colloidal gold assay with centrifuge isolation. In addition, the present application also provides a centrifugal separation and detection device.

An object of the present invention is to provide a paper chromatography chemiluminescence assay method and a combined kit specialized for the method.

The combined kit of paper chromatography and chemiluminescence assay includes an analyte-specific binding substance, a micro particle, a solid phase membrane, a chemiluminescent reactant, a centrifugation device and a luminescence detector.

The chemiluminescent reactant includes at least one of a chemiluminescent enzyme, a chemiluminescent substance, a chemiluminescent enzyme substrate and a luminescence-activating reagent.

The micro particle forms a stable non-specific binding to a protein and/or the chemiluminescent reactant directly and/or by chemical crosslinking.

The solid phase membrane is a membranous substance with a non-specific binding characteristic to a protein.

The centrifugation device centrifugally drives a liquid phase to chromatography and flow on the solid phase membrane.

The analyte-specific binding substance is selected from at least one of substances having a specific binding ability comprising an antigen, an antibody, an avidin and a biotin.

The solid phase membrane of the combined kit of paper chromatography and chemiluminescence includes nitrocellulose membrane, polyvinylidene fluoride membrane, nylon membrane and DEAE cellulose membrane, and the solid phase has a backing on one or both sides. In the present invention, the polyvinylidene fluoride membrane is abbreviated as a PVDF membrane and the DEAE cellulose membrane is a paper membrane prepared by introducing diethylaminoethyl (DEAE) into cellulose molecules, which is also a weak base anion exchange material. The luminescence detector is a chemiluminescence detector and the detector is arranged at one or both sides of the solid phase membrane.

The combined kit of paper chromatography and chemiluminescence assay further includes a cleaning solution. The cleaning solution is a conventional experimental buffer solution containing a surfactant and currently, commonly used surfactants include TWEEN20, TritonX-100 and TritonX-405.

The chemiluminescent enzyme of the combined kit of paper chromatography and chemiluminescence assay includes at least one of horseradish peroxidase, alkaline phosphatase and xanthine oxidase.

Commonly used chemiluminescent substrates of the horseradish peroxide are luminol, isoluminol and its derivatives, such as isoluminol, 4-aminohexyl-N-ethylisoluminol, AHEI and ABEI. Currently, commonly-used products are West Pico chemiluminescent detection substrate, West Dura chemiluminescent detection substrate and West Femto chemiluminescent detection substrate, which are produced by PIERCE Inc.

Commonly used chemiluminescent substrates of the alkaline phosphatase are (adamantane)-1,2-dioxyethane and its derivatives, AMPPD, CDP-STAR and Lumi-Phos 530.

Chemiluminescent substrates of the xanthine oxidase are xanthine, myricetin and quercetin.

The present invention further provides a method for performing a paper chromatography and chemiluminescence assay using the combined kit. The method is selected from any one of the methods A to F as described below.

Method A includes the following steps:

(1) labeling the micro particle simultaneously with the analyte-specific binding substance and the chemiluminescent enzyme;

(2) reacting an analyte-containing sample with the labeled micro particle to achieve a binding between the analyte and the analyte-specific binding substance on the labeled micro particle, and forming a chemiluminescent enzyme-micro particle-analyte-specific binding substance-analyte complex (complex 1);

(3) coating a second analyte-specific binding substance capable of forming specific binding to the analyte on the complex 1 on the solid phase membrane;

(4) chromatographing a liquid phase containing complex 1 to pass through the second analyte-specific binding substance coated on the solid phase membrane, and forming a chemiluminescent enzyme-micro particle-analyte-specific binding substance-analyte-second analyte-specific binding substance complex (complex 2), and then capturing and immobilizing complex 2 onto the solid phase membrane.

(5) cleaning the solid phase membrane to remove the unbound complex 1 and the remaining chemiluminescent enzyme thereon with a cleaning solution using centrifugal chromatography; and

(6) transferring the cleaned solid phase membrane in a solution of the chemiluminescent enzyme substrate for a reaction, and using the luminescence detector to detect a luminescence value generated from the chemiluminescent enzyme substrate through a reaction catalyzed by the chemiluminescent enzyme indirectly immobilized on the solid phase membrane, thereby determining a content of the analyte.

Method B includes the following steps:

(1) labeling the micro particle with the analyte-specific binding substance;

(2) labeling a substance that specifically binds to the analyte-specific binding substance with the chemiluminescent enzyme to form a chemiluminescent enzyme-specific conjugate of the analyte-specific binding substance (a chemiluminescent enzyme marker);

(3) reacting an analyte-containing sample with the labeled micro particle to form a binding between the analyte and the analyte-specific binding substance on the labeled micro particle, thereby forming a micro particle-analyte-specific binding substance-analyte complex (complex 3);

(4) coating the second analyte-specific binding substance on the solid phase membrane;

(5) chromatographing a liquid phase containing complex 3 to pass through the second analyte-specific binding substance coated on the solid phase membrane, and forming a micro particle-analyte-specific binding substance-analyte-second analyte-specific binding substance complex (complex 4), and then capturing and immobilizing complex 4 onto the solid phase membrane.

(6) chromatographing a liquid phase containing the chemiluminescent enzyme marker to pass through complex 4 captured on the solid phase membrane, and forming a chemiluminescent enzyme marker-complex 4 complex (complex 5), through a binding between the chemiluminescent enzyme marker and the analyte-specific binding substance, and then capturing and immobilizing complex 5 onto the solid phase membrane;

(7) cleaning the solid phase membrane to remove the unbound chemiluminescent enzyme marker and the remaining chemiluminescent enzyme thereon with a cleaning solution using centrifugal chromatography; and

(8) transferring the cleaned solid phase membrane in a solution of the chemiluminescent enzyme substrate for a reaction, and using the luminescence detector to detect a luminescence value generated from the chemiluminescent enzyme substrate through a reaction catalyzed by thechemiluminescent enzyme indirectly immobilized on the solid phase membrane, thereby determining a content of the analyte.

Method C includes the following steps:

(1) labeling the micro particle simultaneously with the analyte-specific binding substance and the chemiluminescent enzyme;

(2) reacting an analyte-containing sample with the labeled micro particle to achieve a binding between the analyte and the analyte-specific binding substance on the labeled micro particle, and forming a chemiluminescent enzyme-micro particle-analyte-specific binding substance-analyte complex (complex 1);

(3) labeling the second analyte-specific binding substance with an intermediate A;

(4) coating an intermediate B capable of forming a specific binding to the intermediate A on the solid phase membrane;

(5) reacting a liquid phase containing complex 1 with the second analyte-specific binding substance labeled with the intermediate A to form a chemiluminescent enzyme-micro particle-analyte-specific binding substance-analyte-second analyte-specific binding substance-intermediate A complex (complex 6);

(6) chromatographing a liquid phase containing complex 6 to pass through the intermediate B coated on the solid phase membrane, and forming a chemiluminescent enzyme-micro particle-analyte-specific binding substance-analyte-second analyte-specific binding substance-intermediate A-intermediate B complex (complex 7), and then capturing and immobilizing complex 7 on the solid phase membrane;

(7) cleaning the solid phase membrane to remove the unbound complex 6 and the remaining chemiluminescent enzyme thereon with a cleaning solution using centrifugal chromatography;

(8) transferring the cleaned solid phase membrane in a solution of the chemiluminescent enzyme substrate for a reaction, and using the luminescence detector to detect a luminescence value generated from the chemiluminescent enzyme substrate through a reaction catalyzed by the chemiluminescent enzyme indirectly immobilized on the solid phase membrane, thereby determining a content of the analyte.

Method D includes the following steps:

(1) labeling the micro particle with the analyte-specific binding substance;

(2) labeling a substance that specifically binds to the analyte-specific binding substance with the chemiluminescent enzyme to form a chemiluminescent enzyme-specific binding substance of the analyte-specific binding substance (a chemiluminescent enzyme marker);

(3) reacting an analyte-containing sample with the labeled micro particle to form a binding between the analyte and the analyte-specific binding substance on the labeled micro particle, thereby forming a micro particle-analyte-specific binding substance-analyte complex (complex 3);

(4) labeling the second analyte-specific binding substance with the intermediate A;

(5) coating the intermediate B capable of forming a specific binding to the intermediate A on the solid phase membrane;

(6) reacting a liquid phase containing complex 3 with the second analyte-specific binding substance labeled with the intermediate A to form a micro particle-analyte-specific binding substance-analyte-second analyte-specific binding substance-intermediate A complex (complex 8);

(7) chromatographing a liquid phase containing complex 8 to pass through the intermediate B coated on the solid phase membrane, and forming a micro particle-analyte-specific binding substance-analyte-second analyte-specific binding substance-intermediate A-intermediate B complex (complex 9), and then capturing and immobilizing complex 9 on the solid phase membrane;

(8) chromatographing a liquid phase containing the chemiluminescent enzyme marker to pass through complex 9 captured on the solid phase membrane, and forming a chemiluminescent enzyme-specific conjugate of analyte-specific binding substance-complex 9 complex through a binding with the analyte-specific binding substance (complex 10), and then capturing and immobilizing complex 10 on the solid phase membrane;

(9) cleaning the solid phase membrane to remove the unbound chemiluminescent enzyme marker and the remaining chemiluminescent enzyme thereon with a cleaning solution using centrifugal chromatography; and

(10) transferring the cleaned solid phase membrane in a solution of the chemiluminescent enzyme substrate for a reaction, and using the luminescence detector to detect a luminescence value generated from the chemiluminescent enzyme substrate through a reaction catalyzed by thechemiluminescent enzyme indirectly immobilized on the solid phase membrane, thereby determining a content of the analyte.

Method E includes the following steps:

(1) labeling the micro particle with the analyte-specific binding substance and the chemiluminescent substance;

(2) reacting an analyte-containing sample with the labeled micro particle to form a chemiluminescent substance-micro particle-analyte-specific binding substance-analyte complex (complex 11), through a binding between the analyte and the analyte-specific binding substance on the labeled micro particle;

(3) coating the second analyte-specific binding substance on the solid phase membrane;

(4) chromatographing a solid phase containing complex 11 to pass through the second analyte-specific binding substance coated on the solid phase membrane, and forming a chemiluminescent substance-micro particle-analyte-specific binding substance-analyte-second analyte-specific binding substance complex (complex 12), and capturing and immobilizing complex 12 on the solid phase membrane;

(5) cleaning the solid phase membrane to remove the unbound complex 11 and the remaining chemiluminescent substance with a cleaning solution using centrifugal chromatography; and

(6) transferring the cleaned solid phase membrane in a solution of the luminescence-activating reagent for a reaction, and using the luminescence detector to detect a luminescence value generated from decomposition of the chemiluminescent substance indirectly immobilized on the solid phase membrane, thereby determining a content of the analyte.

Method F includes the following steps:

(1) labeling the micro particle with the analyte-specific binding substance;

(2) labeling a substance that specifically binds to the analyte-specific binding substance with the chemiluminescent substance to form a chemiluminescent substance-specific conjugate of the analyte-specific binding substance (a chemiluminescent substance marker);

(3) reacting an analyte-containing sample with the labeled micro particle to form the micro particle-analyte-specific binding substance-analyte complex (complex 3), through a binding between the analyte and the analyte-specific binding substance on the labeled micro particle;

(4) coating the second analyte-specific binding substance on the solid phase membrane;

(5) chromatographing a solid phase containing complex 3 to pass through the second analyte-specific binding substance coated on the solid phase membrane, and forming the micro particle-analyte-specific binding substance-analyte-second analyte-specific binding substance complex (complex 4), and capturing and immobilizing complex 4 on the solid phase membrane;

(6) chromatographing a solid phase containing the chemiluminescent substance marker to pass through complex 4 captured on the solid phase membrane, and forming a chemiluminescent substance-specific conjugate of the analyte-specific binding substance-complex 4 complex (complex 13), and capturing and immobilizing complex 13 on the solid phase membrane;

(7) cleaning the solid phase membrane to remove the unbound chemiluminescent substance marker and the remaining chemiluminescent substance with a cleaning solution using centrifugal chromatography; and

(8) transferring the cleaned solid phase membrane in a solution of the luminescence-activating reagent for a reaction, and using the luminescence detector to detect a luminescence value generated from decomposition of the chemiluminescent substance indirectly immobilized on the solid phase membrane, thereby determining a content of the analyte.

In the above paper chromatography and chemiluminescence assay methods, the intermediates A and B are independently selected from at least one of substances with a specific binding ability including an antigen, an antibody, an avidin and a biotin.

In the above paper chromatography and chemiluminescence assay methods, liquid phase passing through the solid phase during chromatography is achieved on the centrifugation device and a liquid phase is driven to chromatography and flow on the solid phase membrane using a centrifugal chromatography method.

In the above paper chromatography and chemiluminescence assay methods, a particle size of the micro particle is from 1 to 10,000 nm, specifically from 10 to 3,000 nm, from 20 to 2,000 nm or from 43 to 1,000 nm. Currently, commonly used micro particle includes colloidal gold particle, colloidal selenium particle, colloidal gold magnetic particle, fluorescent microsphere, magnetic micro particle, gold magnetic micro particle, gel micro particle, latex micro particle, plastic microsphere, microsphere silica gel, agarose micro particle, polystyrene micro particle, silicon oxide microsphere, polystyrene microsphere, carboxyl microsphere, chloromethyl microsphere, etc.

In the above paper chromatography and chemiluminescence assay methods, rotational speed of the centrifugation device is controlled by a program, and the rotational speed of the centrifugation device is selected from 200 to 10,000 rpm, specifically from 500 to 5,000 rpm, from 800 to 3,000 rpm or from 800 to 2,000 rpm.

In the above paper chromatography and chemiluminescence assay methods, rotational speed of the centrifugal chromatography is controlled by a program, and the rotational speed of the centrifugal chromatography is selected from 200 to 10,000 rpm, specifically from 500 to 5,000 rpm, from 800 to 3,000 rpm or from 800 to 2,000 rpm.

In the above paper chromatography and chemiluminescence assay methods, a non-enzymatic chemiluminescent substrate (a direct chemiluminescent substance, is used in an immunoassay method for directly labeling an antigen or an antibody with a chemiluminescent agent. Commonly-used chemiluminescent agents mainly include acridinium ester (AE), acridine-amide class and tris (bipyridine) ruthenium, and the acridinium ester is an effective luminescent marker, whose luminescence is achieved with an action of the luminescence-activating reagents such as NaOH and H₂O₂.

Another object of the present invention is to provide a fluorescence immune chromatography assay method with centrifuge isolation and a special combined kit thereof.

The combined kit of fluorescence immune chromatography assay with centrifuge isolation includes an analyte-specific binding substance, a micro particle, a solid phase membrane, a fluorescent substance, a centrifugal device and a fluorescence detector.

The fluorescent substance includes at least one of an organic fluorescent dye and a rare-earth element fluorescent dye.

The micro particle forms a stable non-specific binding to a protein and/or the fluorescent substance directly and/or by chemical crosslinking.

The solid phase membrane is a membranous substance that non-specifically binds to a protein.

The centrifugation device centrifugally drives a liquid phase to chromatography and flow on the solid phase membrane.

The analyte-specific binding substance is selected from at least one of substances having a specific binding ability including an antigen, an antibody, an avidin and a biotin.

In the combined kit of fluorescence immune chromatography assay with centrifuge isolation, the fluorescent substance can be coupled or infiltrated into a microsphere to obtain a fluorescent microsphere. In addition, the fluorescent substance may be a fluorescent dye composed of a single compound or a composite fluorescent dye composed of several compounds, but the fluorescent dye composed of a single compound is preferable, and a fluorescent dye having a strong photostability is preferable. Currently, commonly-used organic fluorescent dyes are fluorescein isothiocyanate (FITC), tetraethyl rhodamine, tetramethyl rhodamine isothiocyanate, phycoerythrin (PE), peridinin chlorophyll protein (PerCP), propidium iodide (PI) and allophycoerythrin (APC), and a commonly-used rare-earth fluorescent material in the rare-earth element fluorescent dye is prepared using alkaline-earth metal sulfides, aluminates, etc. as a luminescent substrate and rare-earth lanthanides (europium, samarium, erbium, etc.) as an activator and a co-activator.

In order to improve discrimination between signal and background, the fluorescent dye of the present invention has a wavelength of 300-1300 nm and the wavelength is preferably 550-800 nm. In addition, a chromatography membrane, a bottom plate and a buckle generally have weak fluorescence intensity in a near-infrared region (600-800 nm), so that a fluorescent dye with a wavelength of 550-800 nm is preferably used to further improve sensitivity. In order to reduce effect on fluorescence signal of the fluorescent dye, the present invention employs a chromatography membrane, a bottom plate and a buckle with a weak florescence intensity, and a fluorescence noise thereof is weak at a wavelength of greater than 550 nm, thereby ensuring a high signal-to-background ratio of fluorescence and distinguishing the signal from the background well, which in turn improves detection sensitivity. Preferably, the bottom plate is white and is attached with a sticker, moreover, the buckle, the chromatography membrane, the bottom plate and the sticker are all free of a fluorescent agent.

In the combined kit of fluorescence immune chromatography assay with centrifuge isolation, the solid phase membrane includes nitrocellulose membrane, polyvinylidene fluoride membrane, nylon membrane and DEAE cellulose membrane, and the solid phase has a backing on one or both sides. In the present invention, the polyvinylidene fluoride membrane is abbreviated as a PVDF membrane and the DEAE cellulose membrane is a paper membrane prepared by introducing diethylaminoethyl (DEAE) into cellulose molecules, which is also a weak base anion exchange material. The luminescence detector is a chemiluminescence detector and the detector is arranged at one or both sides of the solid phase membrane.

The combined kit of paper chromatography and chemiluminescence further includes a cleaning solution. The cleaning solution is a conventional experimental buffer solution containing a surfactant and currently, commonly used surfactants include TWEEN20, TritonX-100 and TritonX-405.

The present invention further provides a paper chromatography and immunofluorescence assay method using the combined kit. The method is selected from any one of the methods A to D as described below.

Method A includes the following steps:

(1) labeling the micro particle with both the analyte-specific binding substance and the fluorescent substance;

(2) reacting an analyte-containing sample with the labeled micro particle to form a fluorescent substance-micro particle-analyte-specific binding substance-analyte complex (complex 1′), through a binding between the analyte and the analyte-specific binding substance on the labeled micro particle;

(3) coating a second analyte-specific binding substance capable of forming a specific binding to the analyte on complex 1′ on the solid phase membrane;

(4) chromatographing a liquid phase containing complex 1′ to pass through the second analyte-specific binding substance coated on the solid phase membrane, and forming a fluorescent substance-micro particle-analyte-specific binding substance-analyte-second analyte-specific binding substance complex (complex 2′), and capturing and immobilizing complex 2′ on the solid phase membrane.

(5) cleaning the solid phase membrane to remove the unbound complex 1′ and the remaining fluorescent substance with a cleaning solution using centrifugal chromatography.

(6) detecting a luminescence value of the fluorescent substance immobilized on cleaned solid phase membrane generated by exciting light using a fluorescence detector to determine a content of the analyte.

Method B includes the following steps:

(1) labeling the micro particle with the analyte-specific binding substance;

(2) labeling a specific conjugate of the analyte-specific binding substance with the fluorescent substance to form a fluorescent substance-specific conjugate of the analyte-specific binding substance (a fluorescent substance marker;

(3) reacting an analyte-containing sample with the micro particle to form a micro particle-analyte-specific binding substance-analyte complex (complex 3′), through a binding between the analyte and the analyte-specific binding substance on the labeled micro particle;

(4) coating the second analyte-specific binding substance on the solid phase membrane;

(5) chromatographing a liquid phase containing complex 3′ to pass through the second analyte-specific binding substance coated on the solid phase membrane, and forming a micro particle-analyte-specific binding substance-analyte-second analyte-specific binding substance complex (complex 4′), and capturing and immobilizing complex 4′ on the solid phase membrane;

(6) chromatographing a liquid phase containing the fluorescent substance to pass through complex 4′ captured on the solid phase membrane, and forming a fluorescent substance marker-complex 4′ complex (complex 5′), through a binding with the analyte-specific binding substance on complex 4′, and capturing and immobilizing complex 5′ on the solid phase membrane;

(7) cleaning the solid phase membrane to remove the unbound fluorescent substance marker and the remaining fluorescent substance with a cleaning solution using centrifugal chromatography; and

(8) detecting a luminescence value of the fluorescent substance immobilized on cleaned solid phase membrane generated by exciting light using a fluorescence detector to determine a content of the analyte.

Method C includes the following steps:

(1) labeling the micro particle simultaneously with the analyte-specific binding substance and the fluorescent substance;

(2) reacting an analyte-containing sample with the labeled micro particle to form the fluorescent substance-micro particle-analyte-specific binding substance-analyte complex (complex 1′), through a binding between the analyte and the analyte-specific binding substance on the labeled micro particle;

(3) labeling the second analyte-specific binding substance with an intermediate A;

(4) coating an intermediate B capable of forming a specific binding to the intermediate A on the solid phase membrane;

(5) reacting a liquid phase containing complex 1′ with the second analyte-specific binding substance labeled with the intermediate A to form a fluorescent substance-micro particle-analyte-specific binding substance-analyte-second analyte-specific binding substance-intermediate A complex (complex 6′).

(6) chromatographing a liquid phase containing complex 6′ to pass through the intermediate B coated on the solid phase membrane, and forming a fluorescent substance-micro particle-analyte-specific binding substance-analyte-second analyte-specific binding substance-intermediate A-intermediate B complex (complex 7′), and capturing and immobilizing complex 7′ on the solid phase membrane;

(7) cleaning the solid phase membrane to remove the unbound complex 6′ and the remaining fluorescent substance with a cleaning solution using centrifugal chromatography; and

(8) detecting a luminescence value of the fluorescent substance immobilized on cleaned solid phase membrane generated by exciting light using a fluorescence detector to determine a content of the analyte.

Method D includes the following steps:

(1) labeling the micro particle with the analyte-specific binding substance;

(2) labeling the specific conjugate of the analyte-specific binding substance with the fluorescent substance to form the fluorescent substance-specific conjugate of the analyte-specific binding substance complex (a fluorescent substance marker);

(3) reacting an analyte-containing sample with the labeled micro particle to form the micro particle-analyte-specific binding substance-analyte complex (complex 3′), through a binding between the analyte and the annlyte-specific conjugate on the labeled micro particle;

(4) labeling the second analyte-specific binding substance with the intermediate A;

(5) coating the intermediate B capable of forming a specific binding to the intermediate A on the solid phase membrane;

(6) reacting a liquid phase containing complex 3′ with the second analyte-specific binding substance labeled with the intermediate A to form a micro particle-analyte-specific binding substance-analyte-second analyte-specific binding substance-intermediate A complex (complex 8′);

(7) chromatographing a liquid phase containing complex 8′ to pass through the intermediate B coated on the solid phase membrane, and forming a micro particle-analyte-specific binding substance-analyte-second analyte-specific binding substance-intermediate A-intermediate B complex (complex 9′), and capturing and immobilizing complex 9′ on the solid phase membrane;

(8) chromatographing a liquid phase containing the fluorescent substance marker to pass through complex 9′ captured on the solid phase membrane, and forming a fluorescent substance-specific conjugate of the analyte-specific binding substance-complex 9′ complex (complex 10′), through a binding with the analyte-specific binding substance, and capturing and immobilizing complex 10′ on the solid phase membrane;

(9) cleaning the solid phase membrane to remove the unbound fluorescent substance marker and the remaining fluorescent substance with a cleaning solution using centrifugal chromatography; and

(10) detecting a luminescence value of the fluorescent substance immobilized on cleaned solid phase membrane generated by exciting light using a fluorescence detector to determine a content of the analyte.

In the above fluorescence immune chromatography assay methods with centrifuge isolation, the solid phase membrane is cleaned using a dry centrifugation cleaning in a centrifugal chromatography manner without a cleaning solution.

In the above fluorescence immune chromatography assay methods with centrifuge isolation, the intermediates A and B are independently selected from at least one of substances with a specific binding ability including an antigen, an antibody, an avidin and a biotin.

In the above fluorescence immune chromatography assay methods with centrifuge isolation, liquid phase passing through the solid phase during chromatography is achieved on the centrifugation device and a liquid phase is driven to chromatography and flow on the solid phase membrane using a centrifugal chromatography method.

In the above methods or the combined kit of fluorescence immune chromatography assay with centrifuge isolation, a particle size of the micro particle is selected from 1 to 10 um, specifically from 10 to 3000 nm, from 20 to 2000 nm or from 35 to 1000 nm. The micro particle forms a stable non-specific binding to a protein and/or the fluorescent substance directly and/or by chemical crosslinking. Currently, commonly-used micro particle includes colloidal gold particle, colloidal selenium particle, colloidal gold magnetic particle, fluorescent microsphere, magnetic micro particle, gold magnetic micro particle, gel micro particle, latex micro particle, plastic microsphere, microsphere silica gel, agarose micro particle, polystyrene micro particle, silicon oxide microsphere, polystyrene microsphere, carboxyl microsphere, chloromethyl microsphere, etc.

In the above combined kit of fluorescence immune chromatography assay with centrifuge isolation, rotational speed of the centrifugation device is controlled by a program, and the rotational speed of the centrifugation device is selected from 200 to 10,000 rpm, specifically from 500 to 5,000 rpm, from 800 to 3,000 rpm or from 800 to 2,000 rpm.

In the above fluorescence immune chromatography assay methods with centrifuge isolation, rotational speed of the centrifugal chromatography is controlled by a program, and the rotational speed of the centrifugal chromatography is selected from 200 to 10,000 rpm, specifically from 500 to 5,000 rpm, from 800 to 3,000 rpm or from 800 to 2,000 rpm.

Yet another object of the present invention is to provide a immune chromatography colloidal gold assay method with centrifuge isolation and a special combined kit thereof.

The combined kit of immune chromatography colloidal gold with centrifuge isolation includes an analyte-specific binding substance, a colloidal gold micro particle, a solid phase membrane, a centrifugal device and a colloidal gold detector.

The colloidal gold micro particle is prepared using a gold solution and forms a stable non-specific binding to a protein directly.

The solid phase membrane is membranous substance that non-specifically binds to a protein.

The centrifugal device centrifugally drives a liquid phase to chromatography and flow on the solid phase membrane.

The analyte-specific binding substance is selected from at least one of substances having a specific binding ability comprising an antigen, an antibody, an avidin and a biotin.

The colloidal gold detector is used to perform a quantitative or semi-quantitative detection through colorimetric analysis of a colloidal gold color.

In the combined kit of immune chromatography colloidal gold assay with centrifuge isolation, a particle size of the colloidal gold micro particle is selected from 10 to 200 nm, specifically from 20 to 150 nm, from 30 to 100 nm or from 30 to 80 nm.

In the combined kit of immune chromatography colloidal gold assay with centrifuge isolation, the solid phase membrane includes nitrocellulose membrane, polyvinylidene fluoride membrane, nylon membrane and DEAE cellulose membrane, and the solid phase has a backing on one or both sides. In the present invention, the polyvinylidene fluoride membrane is abbreviated as a PVDF membrane and the DEAE cellulose membrane is a paper membrane prepared by introducing diethylaminoethyl (DEAE) into cellulose molecules, which is also a weak base anion exchange material.

The combined kit of immune chromatography colloidal gold assay with centrifuge isolation further includes a cleaning solution. The cleaning solution is a conventional experimental buffer solution containing a surfactant and currently, commonly-used surfactants include TWEEN20, TritonX-100 and TritonX-405.

The present invention further provides a method for performing immune chromatography colloidal gold assay with centrifuge isolation using the combined kit, and the method is selected from any one of the following methods A to B.

Method A includes the following steps:

(1) preparing the colloidal gold micro particle with a gold solution;

(2) labeling the colloidal gold micro particle with the analyte-specific binding substance;

(3) reacting an analyte-containing sample with the labeled colloidal gold micro particle to form a colloidal gold micro particle-analyte-specific binding substance-analyte complex (complex 1″), through a binding between the analyte and the analyte-specific binding substance on the labeled colloidal gold micro particle;

(4) coating a second analyte-specific binding substance capable of forming a specific binding to the analyte on complex 1″ on the solid phase membrane;

(5) chromatographing a liquid phase containing complex 1″ to pass through the second analyte-specific binding substance coated on the solid phase membrane, and forming a colloidal gold micro particle-analyte-specific binding substance-analyte-second analyte-specific binding substance complex (complex 2″), and capturing and immobilizing complex 2″ on the solid phase membrane;

(6) cleaning the solid phase membrane to remove the unbound complex 1″ and the remaining colloidal gold with a cleaning solution using centrifugal chromatography; and

(7) detecting a luminescence value of the fluorescent substance immobilized on cleaned solid phase membrane generated by exciting light using a fluorescence detector to determine a content of the analyte.

Method B includes the following steps:

(1) preparing the colloidal gold micro particle with a gold solution;

(2) labeling the colloidal gold micro particle with the analyte-specific binding substance;

(3) reacting an analyte-containing sample with the labeled colloidal gold micro particle to form the colloidal gold micro particle-analyte-specific binding substance-analyte complex (complex 1″), through a binding between the analyte and the analyte-specific binding substance on the labeled colloidal gold micro particle;

(4) labeling the second analyte-specific binding substance with an intermediate A;

(5) coating an intermediate B capable of forming a specific binding to the intermediate A on the solid phase membrane;

(6) reacting a liquid phase containing complex 1″ with the second analyte-specific binding substance labeled with the intermediate A to form a colloidal gold micro particle-analyte-specific binding substance-analyte-second analyte-specific binding substance-intermediate A complex (complex 3″);

(7) chromatographing a liquid phase containing complex3″ to pass through the intermediate B coated on the solid phase membrane, and forming a colloidal gold micro particle-analyte-specific binding substance-analyte-second analyte-specific binding substance-intermediate A-intermediate B complex (complex 4″), and capturing and immobilizing complex 4″ on the solid phase membrane;

(8) cleaning the solid phase membrane to remove the unbound complex 3″ and the remaining colloidal gold with a cleaning solution using centrifugal chromatography; and

(9) detecting a luminescence value of the fluorescent substance immobilized on cleaned solid phase membrane generated by exciting light using a fluorescence detector to determine a content of the analyte.

In the above immune chromatography colloidal gold assay methods with centrifuge isolation, the solid phase membrane is cleaned using a dry centrifugation cleaning in a centrifugal chromatography manner without a cleaning solution (the centrifugal chromatography is directly used to clean centrifugally the solid phase to remove the unbound complex 3 and the remaining colloidal gold).

In the above immune chromatography colloidal gold assay methods with centrifuge isolation, the intermediates are independently selected from at least one of substances with a specific binding ability including an antigen, an antibody, an avidin and a biotin.

In the above immune chromatography colloidal gold assay methods with centrifuge isolation, liquid phase passing through the solid phase during chromatography is achieved on the centrifugation device and a liquid phase is driven to chromatography and flow on the solid phase membrane using a chromatography method with centrifuge isolation.

In the above immune chromatography colloidal gold assay methods with centrifuge isolation, rotational speed of the centrifugation device is controlled by a program, and the rotational speed of the centrifugation device is selected from 200 to 10,000 rpm, specifically from 500 to 5,000 rpm, from 800 to 3,000 rpm or from 800 to 2,000 rpm.

In the above immune chromatography colloidal gold assay methods with centrifuge isolation, rotational speed of the centrifugal chromatography is controlled by a program, and the rotational speed of the centrifugal chromatography is selected from 200 to 10,000 rpm, specifically from 500 to 5,000 rpm, from 800 to 3,000 rpm or from 800 to 2,000 rpm.

Yet another object of the present invention is to provide a centrifugal separation and detection device used in the immune chromatography assay with centrifuge isolation of the invention.

The centrifugal separation and detection device of the present invention includes a sampling member, a solid phase membrane, a centrifugation device and a detector.

The centrifugation device includes a centrifugation rotor driven by a drive motor and a support base, and the centrifugation rotor is supported by the support base.

The sampling member is not directly connected to the centrifugation rotor, and is arranged above, below or outside the centrifugation rotor.

The sampling member includes a liquid phase storage device, an injection tube and an injection pump. The liquid phase storage device is communicated with the injection tube, and a liquid in the liquid phase storage device is driven to enter into the injection tube with the injection pump.

The solid phase membrane is arranged on the centrifugation rotor and a liquid phase sample is loaded directly or indirectly to a proximal side of the solid phase membrane with the injection tube.

The detector is not directly connected to the centrifugation rotor, and may be arranged above, below or outside the centrifugation rotor.

The solid phase membrane of the above centrifugal separation and detection device is arranged in a fixer and the fixer is selected from at least one of support base plate for supporting the solid phase membrane, a lateral flow test strip buckle-like member and a transparent embedded member. The fixer is arranged on the centrifugation rotor and the fixer and the centrifugation rotor both have a detachable structure.

The transparent embedded member of the above centrifugal separation and detection device can cover at least one of upper and lower sides of the solid phase membrane with a transparent material, and an area of the transparent material on the corresponding side of the solid phase membrane is greater than or equal to an area of the solid phase membrane.

The sampling member of the above centrifugal separation and detection device is arranged in one or more rows, and the injection tube can be moved up, down, left, and right.

The centrifugal separation and detection device further includes an ultrasonication device, and the ultrasonication device includes an ultrasonicator, a transducer, an amplitude transformer and an ultrasonic breaking container, and is arranged above, below or outside the centrifugation rotor.

In the centrifugal separation and detection device, a proximal end of the solid phase membrane to the centrifugation rotor is further provided with a liquid adsorption and dispersion member in communication with the solid phase membrane and a telecentric end of the solid phase membrane to the centrifugation rotor is further provided with a liquid collection member in communication with the solid phase membrane. The liquid adsorption and dispersion member acts as a liquid phase loading portion of the injection tube.

The liquid phase storage device of the above centrifugal separation and detection device includes a to-be-tested sample storage device and a detection phase storage device. The to-be-tested sample storage device and the detection phase storage device both are in communication with the injection tube and both are driven by the injection pump.

The liquid phase storage device of the above centrifugal separation and detection device further includes a cleaning solution storage device, and the cleaning solution storage device is in communication with the injection tube and is driven by the injection pump.

The centrifugation rotor of the above centrifugal separation and detection device is of a planar type or an inclined outward from a center type. The centrifugation device is provided with an outer casing.

The centrifugation rotor of the above centrifugal separation and detection device is provided with a solid phase membrane placing device, and the solid phase membrane placing device includes a rotary mobile placing device and/or a groove-shaped pressing placing device. The rotary mobile placing device is a columnar and raised connection device arranged on the centrifugation rotor and a fixer is provided with a hole-shaped member matching the columnar and raised connection device.

The centrifugation device, the sampling member, the ultrasonication device and the detector of the above centrifugal separation and detection device are each provided with a program control device.

The solid phase membrane of the above centrifugal separation and detection device is selected from at least one of a nitrocellulose membrane, a polyvinylidene fluoride membrane, a nylon membrane and a DEAE cellulose membrane.

The liquid adsorption and dispersion member includes at least one of a colloidal gold-labeling adsorption membrane, a fluorescence-labeling antibody adsorption membrane, a chemiluminescence-labeling adsorption membrane, a polyester fiber dispersion membrane and a glass fiber dispersion membrane.

The detector includes any one of detectors of absorbance, fluorescence, chemiluminescence, and image digital processing.

The combined kits described herein and the centrifugal separation and detection device provided by the present invention can be used for detecting an immune product, and specifically, the immune product includes at least one of an antigen, an antibody, an immune cell and a chemical component.

### BRIEF DESCRIPTION OF THE DRAWINGS

FIG. 1 is a schematic view showing the centrifugal separation and detection device of the present invention.

FIG. 2 is a schematic view showing a support base plate for supporting the solid phase membrane.

FIG. 3 is a schematic view of a fixer for the solid phase membrane.

### DETAILED DESCRIPTION OF EMBODIMENTS

Experimental methods used in the following examples are conventional methods unless specified otherwise.

Materials, reagents and the like used in the following examples are commercially available, unless specified otherwise.

### EXAMPLE 1

### Production of Combined Kit of Paper-Chromatography and Chemiluminescence Assay

The combined kit of paper-chromatography and chemiluminescence assay includes: an analyte-specific substance, a micro particle, a solid phase membrane, a chemiluminescent enzyme or a chemiluminescent substance, a centrifugation device, a luminescence detector, and a chemiluminescent enzyme substrate or a luminescence-activating reagent. The combined kit further includes a second analyte-specific substance or an intermediate B, a labeled adsorption membrane pad, a water-absorbing membrane pad and a support base plate.

The following experiments are used to illustrate the detection method of the present invention and effects thereof, but are not intended to limit the scope of the present invention. Experimental methods used in the experiments described below are conventional methods unless specified otherwise. Materials, reagents and the like used in the following experiments are commercially available unless specified otherwise.

Experiment 1 for Paper-Chromatography and Chemiluminescence Assay

Colloidal gold particle was used as a liquid phase carrier, and method A was used for the experiment.

1. Experimental Materials

Anti-human myoglobin polyclonal antibody (Genagates, USA), anti-human myoglobin monoclonal antibody (Genagates, USA), horseradish peroxidase (HRP, SIGMA), spectrophotometer (Shanghai Jinghua Instrument Co., Ltd., 752 UV-Vis Spectrophotometer), human myoglobin (Sigma-Aldrich), BioFlow imprinter (IMAGENE, USA), Index siltter (A-point, USA), DBF-900 sealing machine (Wenzhou Jiangnan Packaging Factory), ACBO dehumidifier (Jiangsu Wuxi Aobo Dehumidifier Company), desk centrifuge (Eppendoff Company, USA), bovine serum albumin (abbreviated as BSA, SIGMA), nitrocellulose membrane (AE 99, provided by Genagates, USA), polyester cellulose membrane (Reemay 2033, Alstrom, USA), water-absorbing membrane pad (Grade 470, S&S Company, USA), chloroauric acid (SIGMA), chemiluminescence detector (Promega, Glomax Multi JR Detection System), West Pico luminescent reagent (Thermo Scientific).

In the present experiment, the anti-human myoglobin polyclonal antibody was used as a second analyte-specific substance; the anti-human myoglobin monoclonal antibody was used as an analyte-specific substance; the horseradish peroxidase was used as a chemiluminescent enzyme of a chemiluminescent reactant; the human myoglobin was used as an analyte; the desk centrifuge was used as a centrifugation device; the nitrocellulose membrane was used as a solid phase membrane; the chloroauric acid was a material for preparing the micro particle; the chemiluminescence detector was a luminescence detector; and West Pico chemiluminescence detection substrate was a chemiluminescent enzyme substrate of the chemiluminescent reactant.

2. Experimental Methods

Preparation of human myoglobin solutions:

A human myoglobin solution of known concentration was diluted into 0.1, 1.0, 10, 100, 500, 1000 ng/mL of human myoglobin solutions, respectively, with a sample dilution buffer (1% BSA, 100 mM glycine, 50 mM PBS and 150 mM NaCl, pH 7.4).

Colloidal gold micro particle labeling:

10 mL of pure water was heated under stirring. When the water was boiled, 500 μL of a 10% chloroauric acid solution was added to produce a mixture. The mixture was boiled for 5 minutes and added with 500 μL of a 12% trisodium citrate solution to produce a blend. The blend was kept boiling under stirring for 10 minutes and then naturally cooled to room temperature, thereby producing a colloidal gold micro particle solution with a particle size of 50 nm. 10 mL of the colloidal gold micro particle solution was added to a beaker and adjusted to pH 8.3 with 10% potassium carbonate to produce a mixed solution. The mixed solution was then quickly added with 100 μg of anti-human myoglobin monoclonal antibody and 100 μg of horseradish peroxidase to a final concentration of 10 μg/mL each. The beaker was then shaken to allow a uniform mix of the liquid therein. The beaker was kept at room temperature for 30 minutes. Then 100 μL 10% BSA solution was quickly added to the beaker to a final concentration of 1%, and the beaker was kept under shaking and at room temperature for 30 minutes. The reaction in the beaker was then centrifuged at 12,000 rpm for 20 minutes, and the resulting supernatant was carefully sucked out. The precipitate was suspended with 5 mL of 50 mM phosphate (PBS) buffer (pH 7.4) to produce a suspension. The suspension was then centrifuged at 12,000 rpm for 20 minutes, and the supernatant was sucked out. And the precipitate was dissolved in 1.0 mL of phosphate buffer containing 1% BSA and 3% sucrose to produce a solution. The solution was stored in the dark at 4° C. for use.

Preparation of colloidal gold micro particle-labeled adsorption membrane:

A polyester cellulose membrane pretreatment liquid with pH 7.4 containing 0.5% PVA (i.e., polyvinyl alcohol), 50 mM PBS solution, 0.5% BSA and 0.88% NaCl, was prepared. A to-be-treated polyester cellulose membrane was soaked in the pretreatment solution at room temperature for 1 hour. The membrane was transferred, dried at 37° C. and sealed for later use, or directly used as a dispersion membrane. The colloidal gold particle-labeled antibody solution was diluted with a colloidal buffer (1% BSA, 3% sucrose, 50 mM PBS, pH 7.4) to an OD₅₃₀ of 30. The imprinter was started and loaded with an antibody, and pressurized nitrogen was turned on. The polyester cellulose membrane was used to perform membrane imprinting with an imprinting conditions set as follows: a moving speed of airbrush was 30 mm/sec, and a liquid propelling speed was 5.0 μL/cm. The imprinted membrane was dried in a drying oven and at 37° C. for 6 hours, and was then preserved in a sealed bag containing desiccant for later use.

Preparation of polyclonal antibody-imprinted membrane:

The anti-human myoglobin polyclonal antibody solution was diluted to a concentration of 1 mg/mL with 50 mM phosphate buffer (pH 7.4). The imprinter was started and loaded with the antibody solution. A PVC sheet (i.e. polyvinyl chloride sheet) attached with a nitrocellulose membrane was used to perform membrane imprinting. The imprinting conditions were set as follows: a moving speed of airbrush is 30 mm/s, and a liquid propelling speed is 1.5 μL/cm. The imprinted membrane was dried at 37° C. in a drying oven for 6 hours, and was then preserved in a drying container containing desiccant for later use.

Method for assembling semi-finished product:

A dehumidifier was started to reduce the humidity in an operating room to less than 25%. A water-absorbing paper membrane pad and a colloidal gold micro particle-labeled adsorption membrane were pasted respectively on each end of the polyclonal antibody-imprinted membrane, and a surface thereof was sealed with an adhesive tape. A well-pasted detection sheet was placed on a slitter and cut into 3.5 mm test strips. The test strip was placed in an aluminum foil sealed bag containing desiccant, which was sealed by a sealing machine and labeled.

Test group:

The test strip prepared above was placed in a centrifugation rotor with its colloidal gold particle-labeled absorption membrane facing upwards. The test stripe was added with 80 μL of each of the prepared human myoglobin solutions of different concentrations on its colloidal gold particle-labeled absorption membrane, and was allowed for a standing for 2 minutes. The test strip was then centrifuged at 1,000 rpm for 1 minute, and was added with 80 μL of 50 mM PBS (pH 7.4) containing 0.05% Tween-20 on its colloidal gold particle-labeled absorption membrane. The test strip was centrifuged at 2,000 rpm for 30 seconds for a cleaning, and the cleaning was repeated once. The cleaned test strip was cut to obtain a polyclonal antibody-imprinted membrane detection line which was placed in a transparent test tube. The polyclonal antibody-imprinted membrane detection line was added with a West Pico chemiluminescence detection substrate and allowed for a standing for 5 minutes. A luminescence value was read by a chemiluminescence detector for 6 seconds. The experiment was repeated three times, and the results were averaged and used to plot a concentration-luminescence curve and calculate a correlation coefficient thereof.

Control group:

The test strip prepared above was placed on a table, and was added with 80 μL of each of the prepared human myoglobin solutions of different concentrations on its colloidal gold micro particle-labeled adsorption membrane, followed by a standing. After all red color colloidal gold micro particle marker on the colloidal gold micro particle-labeled adsorption membrane flowed completely into the nitrocellulose membrane via chromatography, the colloidal gold micro particle-labeled adsorption membrane was added with 80 μL of 50 mM PBS (pH 7.4) containing 0.05% Tween-20 followed by a standing to allow a complete flow of the liquid into the nitrocellulose membrane. The test strip was cleaned twice. The cleaned test strip was cut to obtain a polyclonal antibody-imprinted membrane detection line which was placed in a transparent test tube. The polyclonal antibody-imprinted membrane detection line was added with a West Pico chemiluminescence detection substrate and allowed for a standing for 5 minutes. A luminescence value was read with the chemiluminescence detector for 6 seconds. The experiment was repeated three times, and the results were averaged and used to plot a concentration-luminescence curve and calculate a correlation coefficient thereof.

3. Experimental Results

In the present experiment, the colloidal gold particle was used as a liquid phase reaction carrier, and method A was used for the experiment. As a result, the average detection time of the test strip of the test group was 4.6 minutes, while that of the control group (without centrifugation processing) was 47 minutes. The results of the test group showed a good correlation between concentration and luminescence value with a correlation coefficient r of 0.995. While the results of the control group were below 100 ng/mL and substantially no correlation between concentration and luminescence value was found with a correlation efficient of 0.776, P<0.05, which was mainly resulted from an undesirable cleaning effect of the chemiluminescent enzyme due to a non-specific binding on the solid phase membrane, thereby resulting in a high background. The present invention has significantly better test results than that obtained using the assay without centrifugation processing, which indicated that the linearity and accuracy of the detection were improved using the technique of the present invention when compared with the prior art. The experimental results are shown in Table 1.

TABLE 1
Experimental results (luminescence value) of the paper-chromatography
and chemiluminescence assay using method A

| | Concentration (ng/mL) | | | | | |
|---|---|---|---|---|---|---|
| | 0.1 | 1.0 | 10 | 100 | 1000 | r value |
| Test group | 13408 | 51590 | 151777 | 612041 | 3982240 | 0.995 |
| Control group | 1221058 | 1321243 | 1282717 | 1453818 | 4223113 | 0.776 |

Experiment 2 for Paperchromatography and Chemiluminescence Assay

Colloidal gold micro particle was used as a liquid phase reaction carrier, and method B was used for the experiment.

1. Experimental Materials

Alkaline phosphatase (ALP, Roche), goat anti-mouse IgG polyclonal antibody (Genagates, USA), APLS luminescent reagent (Zhengzhou Innosep Biosciences Co., Ltd), and other materials were referred to Experiment 1.

In the present experiment, the alkaline phosphatase was used as a chemiluminescent enzyme of a chemiluminescent reactant; the goat anti-mouse IgG polyclonal antibody specifically binds to the analyte-specific substance; the APLS luminescent reagent was used as a chemiluminescent reaction substrate of the chemiluminescent reactant; and others were referred to Experiment 1.

2. Experimental Methods

Preparation of human myoglobin solutions: Referring to Experiment 1.

Alkaline phosphatase-labeling of goat anti-mouse IgG polyclonal antibody

Modified sodium periodate method was adopted. 1 mg of alkaline phosphatase was dissolved in 200 μL of 0.3 M NaHCO₃ buffer (pH 8.0) to produce a solution. The solution was then added with 1 mL of 0.06 M iodine. The reaction was gently stirred at room temperature in the dark for 0.5 hour. The reaction was then added with 1 mL of 0.16 M ethylene glycol and gently stirred for 1 hour at room temperature to terminate oxidation. The oxidized product was dialyzed overnight against a 0.01 M pH 9.5 carbonate buffer at 4° C. The dialyzed product was then added with 1 mL of carbonate buffer containing 0.5 mg of antibody and gently stirred at room temperature for 3 hours in the dark to produce a mixture. The mixture was then added with 5 mg of NaHB₄ and placed at 4° C. overnight. The reaction was then dialyzed against 0.01 M PBS (pH 7.2) at 4° C. for 24 hours. The dialyzed reaction was then centrifuged at 4,000 rpm and 4° C. for 30 minutes. The precipitate was discarded and the supernatant was mixed with an equal volume of glycerin, and was preserved at −20° C.

Colloidal gold micro particle labeling:

10 mL of colloidal gold solution was added to a beaker and adjusted to pH 8.3 with 10% potassium carbonate to produce a mixture. The mixture was then quickly added with 100 μg of anti-human myoglobin monoclonal antibody to a final concentration of 10 μg/mL to produce a blend. The beaker was shaken to allow a uniform mix of the blend. Other procedures are referred to Experiment 1.

Preparation of colloidal gold particle-labeled adsorption membrane: referring to Experiment 1.

Preparation of polyclonal antibody-imprinted membrane: referring to Experiment 1.

Method for assembling semi-finished product: referring to Experiment 1.

Test group:

The test strip prepared above was placed in a centrifugation rotor with its colloidal gold particle-labeled absorption membrane facing upwards. The test stripe was added with 80 μL of each of the prepared human myoglobin solutions of different concentrations on its colloidal gold particle-labeled absorption membrane, and was allowed for a standing for 2 minutes. The test strip was then centrifuged at 1,000 rpm for 1 minute. The centrifuged test strip was added with 80 μL of the alkaline phosphatase-labeled goat anti-mouse IgG polyclonal antibody solution on its colloidal gold particle-labeled absorption membrane and allowed for a standing for 2 minutes. The test strip was then centrifuged at 1,000 rpm for 1 minute and was added with 80 μL of 50 mM PBS (pH 7.4) containing 0.05% Tween-20 on its colloidal gold particle-labeled absorption membrane. The test strip was then centrifuged at 2,000 rpm for 30 seconds for cleaning, and the cleaning was repeated once. The test strip was then cut to obtain a polyclonal antibody-imprinted membrane detection line which was placed in a transparent test tube. The polyclonal antibody-imprinted membrane detection line was added with the APLS chemiluminescence detection substrate and allowed for a standing for 1 minute. A luminescence value of the resulting product was read by the chemiluminescence detector for 6 seconds. The experiment was repeated three times, and the results were averaged and used to plot a concentration-luminescence curve and calculate a correlation coefficient thereof.

Control group:

The test strip prepared above was placed on a table, and was dripped with 80 μL of each of the prepared human myoglobin solutions of different concentrations on its colloidal gold particle-labeled adsorption membrane followed by a standing. After a complete flow of the red-color colloidal gold micro particles on the colloidal gold micro particle-labeled adsorption membrane into the nitrocellulose membrane via chromatography, the colloidal gold micro particle-labeled adsorption membrane was added with 80 μL of the alkaline phosphatase-labeled goat anti-mouse IgG polyclonal antibody solution followed by a standing to allow the liquid flow completely into the nitrocellulose membrane. The colloidal gold particle-labeled adsorption membrane was then dripped with 80 μL of 50 mM PBS (pH 7.4) containing 0.05% Tween-20 followed by a standing to allow the liquid to flow completely into the nitrocellulose membrane for cleaning. The cleaning was repeated once. The test strip was cut to obtain a polyclonal antibody-imprinted detection line which was placed in a transparent test tube. The polyclonal antibody-imprinted membrane detection line was added with the APLS chemiluminescence detection substrate followed by a standing for 1 minute. A luminescence value of the resulting product was read by the chemiluminescence detector for 6 seconds. The experiment was repeated three times, and the results were averaged and used to plot a concentration-luminescence curve and calculate a correlation coefficient thereof.

3. Experimental Results

In the present experiment, the colloidal gold particle was used as a liquid phase reaction carrier, and method B was used for the experiment. As a result, the average detection processing time of the test strip of the test group of the present invention was 7.8 minutes, while that of the control group (without centrifugation processing) was 62 minutes. The results of the test group of the present invention showed a good correlation between concentration and luminescence value with a correlation coefficient r of 0.994. While the results of the control group were below 100 ng/mL and substantially no correlation between concentration and luminescence value was found with a correlation coefficient r of 0.807, P<0.05, which was mainly resulted from an undesirable cleaning effect of the chemiluminescent enzyme due to a non-specific binding on the solid phase membrane, thereby resulting in a high background. The present invention has significantly better test results than that obtained using the assay without centrifugation processing, which indicated that the linearity and accuracy of the detection were improved using the technique of the present invention when compared with the prior art. The experimental results are shown in Table 2.

TABLE 2
Experimental results (luminescence value) of the paper-chromatography
and chemiluminescence assay using method B

| | Concentration (ng/mL) | | | | | |
|---|---|---|---|---|---|---|
| | 0.1 | 1.0 | 10 | 100 | 1000 | r value |
| Test group | 15231 | 62137 | 182134 | 620341 | 4122450 | 0.994 |
| Control group | 1021856 | 1131246 | 1187652 | 1298713 | 3956718 | 0.807 |

Experiment 3 for Paper-Chromatography and Chemiluminescence Assay

Colloidal gold particle was used as a liquid phase reaction carrier, and method C was used for the experiment.

1. Experimental Materials

NHS-activated biotin (SIGMA), avidin (SIGMA), and other materials were referred to Experiment 1.

In the present experiment, the biotin was used as an intermediate A; the avidin was used as an intermediate B; and others were referred to Experiment 1.

2. Experimental Methods

Preparation of human myoglobin solutions: referring to Experiment 1.

Biotin labeling of anti-human myoglobin polyclonal antibody:

The anti-human myoglobin polyclonal antibody was dialyzed against a 0.1 M pH 9.5 sodium carbonate buffer overnight, and adjusted to a final concentration of 2 mg/mL to produce an antibody solution. 20 mg of the NHS-activated biotin was dissolved in 1 mL of dimethylformamide to produce a solution. 50 μL of the solution was added to the above antibody solution for a reaction at room temperature for 4 hours. The resulting product was dialyzed overnight against PBS buffer and preserved at −20° C.

Colloidal gold particle labeling: referring to Experiment 1.

Preparation of colloidal gold particle-labeled absorption membrane:

The colloidal gold particle-labeled antibody solution was diluted with a colloidal buffer (1% BSA, 3% sucrose, 50 mM PBS, pH 7.4) to an OD₅₃₀ of 30. The diluted solution was then added with the biotin-labeled anti-human myoglobin polyclonal antibody at 10 μg/mL and was mixed uniformly. Other procedures are referred to Experiment 1.

Preparation of avidin-imprinted membrane:

The avidin solution was diluted to a concentration of 1 mg/mL with a 50 mM phosphate buffer (pH 7.4). The imprinter was started and loaded with the diluted avidin solution. Other procedures are referred to Experiment 1.

Method for assembling semi-finished product:

The dehumidifier was started to reduce the humidity in the operating room to less than 25%. A water-absorbing paper membrane pad and a colloidal gold particle-labeled adsorption membrane were pasted respectively on each end of the avidin-imprinted membrane. Other procedures are referred to Experiment 1.

Test group:

The test strip prepared above was placed in a centrifugation rotor with its colloidal gold particle-labeled absorption membrane facing upwards. The test stripe was added with 80 μL of each of the prepared human myoglobin solutions of different concentrations on its colloidal gold particle-labeled absorption membrane followed by a standing for 2 minutes. The test strip was then centrifuged at 1,000 rpm for 1 minute, and was added with 80 μL of 50 mM PBS (pH 7.4) containing 0.05% Tween-20 on its colloidal gold particle-labeled absorption membrane. The test strip was then centrifuged at 2,000 rpm for 30 seconds for cleaning, and the cleaning was repeated once. The test strip was cut to obtain an avidin-imprinted membrane detection line, which was placed in a transparent test tube. The avidin-imprinted membrane detection line was added with a West Pico chemiluminescence detection substrate followed by a standing for 5 minutes. A luminescence value of the resulting product was read by the chemiluminescence detector for 6 seconds. The experiment was repeated three times, and the results were averaged and used to plot a concentration-luminescence curve and calculate a correlation coefficient thereof.

Control group:

The test strip prepared above was placed on a table, and was added with 80 μL of each of the prepared human myoglobin solutions of different concentrations on its colloidal gold particle-labeled adsorption membrane followed by a standing. After a complete flow of the red-color colloidal gold micro particles into the nitrocellulose membrane via chromatography, the colloidal gold micro particle-labeled adsorption membrane was added with 80 μL of 50 mM PBS (pH 7.4) containing 0.05% Tween-20 followed by an standing to allow the liquid flow completely into the nitrocellulose membrane (for cleaning). The cleaning was repeated once. The test strip was cut to obtain an avidin-imprinted detection line, which was placed in a transparent test tube. The avidin-imprinted membrane detection line was added with the West Pico chemiluminescence detection substrate followed by a standing for 5 minutes. A luminescence value of the resulting product was read by the chemiluminescence detector for 6 seconds. The experiment was repeated three times, and the results were averaged and used to plot a concentration-luminescence curve and calculate a correlation coefficient thereof. 3. Experimental Results

In the present experiment, the colloidal gold particle was used as a liquid phase reaction carrier, and method C was used to perform the experiment. As a result, the average detection processing time of the test strip of the test group of the present invention was 4.6 minutes, while that of the control group (without centrifugation processing) was 42 minutes. The test data of the test group of the present invention showed a good correlation between concentration and luminescence value. Specifically, a reaction plateau was reached at 500 ng/mL, and a correlation coefficient r was 0.987. While the results of the control group were below 100 ng/mL and substantially no correlation between concentration and luminescence value was found with a correlation coefficient r of 0.866, P<0.05, which was mainly resulted from an undesirable cleaning effect of the chemiluminescent enzyme due to a non-specific binding on the solid phase membrane, thereby resulting in a high background. The present invention has significantly better test results than that obtained using the assay without centrifugation processing, which indicated that the linearity and accuracy of the detection were improved using the technique of the present invention when compared with the prior art. The experimental results are shown in Table 3.

TABLE 3
Experimental results (luminescence value) of the paper-chromatography
and chemiluminescence assay using method C

| | Concentration (ng/mL) | | | | | |
|---|---|---|---|---|---|---|
| | 0.1 | 1.0 | 10 | 100 | 1000 | r value |
| Test group | 35236 | 92718 | 282196 | 2200345 | 4011368 | 0.987 |
| Control group | 1132456 | 1098213 | 1125768 | 1932661 | 4251784 | 0.866 |

Experiment 4 for Paper-Chromatography and Chemiluminescence Assay

Colloidal gold particle was used as a liquid phase reaction carrier, and method D was used to perform the experiment.

1. Experimental Materials

NHS-activated biotin (SIGMA), avidin (SIGMA), alkaline phosphatase (ALP, Roche), goat anti-mouse IgG polyclonal antibody (Genagates, USA), APLS luminescent reagent (Zhengzhou Innosep Biosciences Co., Ltd), and other materials were referred to Experiment 1.

In the present experiment, the alkaline phosphatase was used as a chemiluminescent enzyme of a chemiluminescent reactant; the goat anti-mouse IgG polyclonal antibody was used as a specific conjugate of an analyte-specific binding substance; the biotin was used as an intermediate A; the avidin was used as an intermediate B; the APLS luminescent reagent was used as a chemiluminescent reaction substrate of the chemiluminescent reactant; and others were referred to Experiment 1.

2. Experimental Methods

Preparation of human myoglobin solutions: referring to Experiment 1.

Alkaline phosphatase-labeling of goat anti-mouse IgG polyclonal antibody: referring to Experiment 2.

Biotin labeling of anti-human myoglobin polyclonal antibody: referring to Experiment 3.

Colloidal gold particle: referring to Experiment 2.

Preparation of colloidal gold particle-labeled absorption membrane: referring to Experiment 3.

Preparation of avidin-imprinted membrane: referring to Experiment 3.

Method for assembling semi-finished product: referring to Experiment 3.

Test group:

The test strip prepared above was placed in a centrifugation rotor with its colloidal gold particle-labeled absorption membrane facing upwards. The test stripe was added with 80 μL of each of the prepared human myoglobin solutions of different concentrations on its colloidal gold particle-labeled absorption membranefollowed by a standing for 2 minutes. The test strip was then centrifuged at 1,000 rpm for 1 minute, and was added with 80 μL of the alkaline phosphatase-labeled goat anti-mouse IgG polyclonal antibody solution on its colloidal gold particle-labeled absorption membrane followed by a standing for 2 minutes. The test strip was then centrifuged at 1,000 rpm for 1 minute and was added with 80 μL of 50 mM PBS (pH 7.4) containing 0.05% Tween-20 on its colloidal gold particle-labeled absorption membrane. The test strip was then centrifuged at 2,000 rpm for 30 seconds for a cleaning, and the cleaning was repeated once. The test strip was cut to obtain an avidin-imprinted detection line, which was placed in a transparent test tube. The avidin-imprinted membrane detection line was added with an APLS chemiluminescence detection substrate followed by a standing for 1 minute. A luminescence value of the resulting product was read by the chemiluminescence detector for 6 seconds. The experiment was repeated three times, and the results were averaged and used to plot a concentration-luminescence curve and calculate a correlation coefficient thereof.

Control group:

The test strip prepared above was placed on a table, and was added with 80 μL of each of the prepared human myoglobin solutions of different concentrations on its colloidal gold particle-labeled adsorption membrane followed by a standing. After a complete flow of the red-color colloidal gold micro particles on the colloidal gold micro particle-labeled adsorption membrane into the nitrocellulose membrane via chromatography, the colloidal gold micro particle-labeled adsorption membrane was added with 80 μL of the alkaline phosphatase-labeled goat anti-mouse IgG polyclonal antibody solution followed by a standing to allow the liquid flow completely into the nitrocellulose membrane. The colloidal gold micro particle-labeled adsorption membrane was then added with 80 μL of 50 mM PBS (pH 7.4) containing 0.05% Tween-20 followed by a standing to allow the liquid flow completely into the nitrocellulose membrane for cleaning. The cleaning was repeated once. The test strip was cut to obtain an avidin-imprinted membrane detection line, which was placed in a transparent test tube. The avidin-imprinted membrane detection line was added with the APLS chemiluminescence detection substrate followed by a standing for 1 minute. A luminescence value of the resulting product was read by the chemiluminescence detector for 6 seconds. The experiment was repeated three times, and the results were averaged and used to plot a concentration-luminescence curve and calculate a correlation coefficient thereof. 3. Experimental Results

In the present experiment, the colloidal gold micro particle was used as a liquid phase reaction carrier, and method D was used for the experiment. As a result, the average detection processing time of the test strip of the test group of the present invention was 7.9 minutes, while that of the control group (without centrifugation processing) was 58 minutes. The test data of the test group of the present invention showed a good correlation between concentration and luminescence value with a correlation coefficient r of 0.987. While the results of the control group were below 100 ng/mL and substantially no correlation between concentration and luminescence value was found with a correlation coefficient r of 0.841, P<0.05, which was mainly resulted from an undesirable cleaning effect of the chemiluminescent enzyme due to a non-specific binding to the solid phase membrane, thereby resulting in a high background. The present invention has significantly better test results than those obtained using the assay without centrifugation processing, which indicated that the linearity and accuracy of the detection were improved using the technique of the present invention when compared to the prior art. Meanwhile, the addition of the intermediate produces a signal amplification effect, which increases the reaction intensity of each concentration. The experimental results are shown in Table. 4.

TABLE 4
Experimental results (luminescence value) of the paper-chromatography
and chemiluminescence assay using method D

| | Concentration (ng/mL) | | | | | |
|---|---|---|---|---|---|---|
| | 0.1 | 1.0 | 10 | 100 | 1000 | r value |
| Test group | 42315 | 87964 | 302210 | 1989321 | 3897652 | 0.987 |
| Control group | 1200761 | 1196751 | 1123970 | 1878652 | 4022306 | 0.841 |

Experiment 5 for Paper-Chromatography and Chemiluminescence Assay

Fluorescent microsphere was used as a liquid phase reaction carrier, and method E was used to perform the experiment. 1. Experimental Materials

Fluorescent microspheres (Shanghai Jieyi Biotech), Trehalose (SIGMA), nitrocellulose membrane (Millipore), EDC (PIERCE), NHS (PIERCE), NHS-activated acridinium ester (acridinium NHS ester, Shanghai Xinle Biotech), luminescence-starting reagent (50 μL of 26.8 mM H₂O₂ solution and 300 μL of 0.2 M NaOH solution, mixed when used), and other materials were referred to Experiment 1.

In the present experiment, the fluorescent microsphere was a micro particle; the NHS-activated acridinium ester was used as a chemiluminescent substance of a chemiluminescent reactant; the luminescence-activating reagent was used as a luminescence-starting reagent of the chemiluminescent reactant; and others were referred to Experiment 1.

2. Experimental Methods

Preparation of human myoglobin solutions: referring to Experiment 1.

Acridinium ester-labeling of anti-human myoglobin monoclonal antibody:

5 mg of anti-human myoglobin monoclonal antibody was dissolved in 2 mL of a 0.2 M aqueous sodium bicarbonate solution (pH 8.3) to produce a mixture. 20 mg/mL NHS-activated acridinium ester was then mixed with the mixture at a ratio of 8 (μL):1 (mg) to the anti-human myoglobin monoclonal antibody for a reaction at 4° C. under shaking for 4 hours. The resulting product was then dialyzed against PBS buffer (pH 7.4) overnight to remove free acridinium ester. The dialyzed product was preserved at 4° C. for use.

Fluorescent microsphere labeling:

0.5 mL of the microsphere was cleaned with 0.1 M phosphate buffer (pH 7.2) and centrifuged at 13,000 rpm. Such process was repeated three times. The cleaned microsphere was redissolved with 0.1 M phosphate buffer (pH 7.2) to 1 mL to produce a blend. The blend was then added with 200 μg of the acridinium ester-labeled anti-human myoglobin monoclonal antibody and mixed uniformly. The reaction was then added with 250 μL of 40 mg/mL EDC solution and 250 μL of 40 mg/mL NHS solution, and mixed uniformly for a reaction at room temperature for 60 minutes. The resulting product was then added with 20 mg of BSA, mixed uniformly, and reacted at room temperature for 60 minutes. The resulting product was then centrifuged, and the supernatant was discarded. The microsphere obtained was further cleaned with 0.05 M Tris (pH 7.6) and centrifuged 4 times. The resulting precipitate was redissolved to 10 mL with 0.5% trehalose, 1% BSA, 0.05 M Tris (pH 7.6), and was preserved at 4° C. in the dark for use.

Preparation of fluorescent microsphere-labeled absorption membrane: The fluorescent microsphere-labeled antibody solution was diluted 2 times with 0.1 M phosphate buffer solution (pH 7.2). Other procedures were referred to Experiment 1.

Preparation of polyclonal antibody-imprinted membrane: referring to Experiment 1.

Method for assembling semi-finished product: referring to Experiment 1.

Test group:

The test strip prepared above was placed in a centrifugation rotor with its fluorescent microsphere-labeled absorption membrane facing upwards. The test stripe was added with 80 μL of each of the prepared human myoglobin solutions of different concentrations on its fluorescent microsphere-labeled absorption membrane followed by a standing for 2 minutes. The test strip was then centrifuged at 1,000 rpm for 1 minute, and was added with 80 μL of 50 mM PBS (pH 7.4) containing 0.05% Tween-20 on its fluorescent microsphere-labeled absorption membrane. The test strip was then centrifuged at 2,000 rpm for 30 seconds for cleaning, and the cleaning was repeated once. The test strip was then cut to obtain a polyclonal antibody-imprinted membrane detection line, which was placed in a transparent test tube. The test tube was then placed in a chemiluminescence detector and added with the luminescence-activating reagent with a delay time set to 0.30 seconds and a luminescence integration time set to 10 seconds. The experiment was repeated three times, and the results were averaged and used to plot a concentration-luminescence curve and calculate a correlation coefficient thereof.

Control group:

The test strip prepared above was placed on a table, and was added with 80 μL of each of the prepared human myoglobin solutions of different concentrations on its fluorescent microsphere-labeled adsorption membrane followed by a standing. After the liquid on the fluorescent microsphere-labeled adsorption membrane flowed completely into the nitrocellulose membrane via chromatography, the fluorescent microsphere-labeled adsorption membrane was added with 80 μL of 50 mM PBS (pH 7.4) containing 0.05% Tween-20 followed by a standing to allow the liquid flow completely into the nitrocellulose membrane for cleaning. The cleaning was repeated once. The test strip was then cut to obtain a polyclonal antibody-imprinted membrane detection line, which was placed in a transparent test tube. The test tube was then placed in the chemiluminescence detector and added with the luminescence-activating reagent with a delay time set to 0.30 seconds and a luminescence integration time set to 10 seconds. The experiment was repeated three times, and the results were averaged and used to plot a concentration-luminescence curve and calculate a correlation coefficient thereof.

3. Experimental Results

In the present experiment, the fluorescent microsphere was used as a liquid phase reaction carrier, and method E was used for the experiment. As a result, the average detection processing time of the test strip of the test group of the present invention was 4.7 minutes, while that of the control group (without centrifugation processing) was 48 minutes. The test data of the test group of the present invention showed a good correlation between concentration and luminescence value with a correlation coefficient r of 0.981. While the results of the control group were below 100 ng/mL and substantially no correlation between concentration and luminescence value was found with a correlation coefficient r of 0.908, P<0.05, which was mainly resulted from an undesirable cleaning effect of the chemiluminescent enzyme due to a non-specific binding to the solid phase membrane, thereby resulting in a high background. The present invention has significantly better test results than those obtained using the assay without centrifugation processing, which indicated that the linearity and accuracy of the detection were improved using the technique of the present invention when compared to the prior art. The experimental results are shown in Table. 5.

TABLE 5
Experimental results (luminescence value) of the paper-chromatography
and chemiluminescence assay using method E

| | Concentration (ng/mL) | | | | | |
|---|---|---|---|---|---|---|
| | 0.1 | 1.0 | 10 | 100 | 1000 | r value |
| Test group | 62564 | 110325 | 338521 | 2357819 | 11892567 | 0.981 |
| Control group | 1105827 | 1096251 | 1223158 | 5817652 | 12510981 | 0.908 |

Experiment 6 for Paper-Chromatography and Chemiluminescence Assay

Fluorescent microsphere was used as a liquid phase reaction carrier, and method F was used for the experiment.

1. Experimental Materials

Goat anti-mouse IgG polyclonal antibody (Genagates, USA); and other materials were referred to Experiment 5.

In the present experiment, the goat anti-mouse IgG polyclonal antibody was used as a specific conjugate of an analyte-specific binding substance, and others were referred to Experiment 5.

2. Experimental Methods

Preparation of human myoglobin solutions: referring to Experiment 1.

Acridinium ester-labeling of goat anti-mouse IgG polyclonal antibody:

5 mg of anti-human myoglobin monoclonal antibody was dissolved in 2 mL of a 0.2 M aqueous sodium hydrogencarbonate solution (pH 8.3) to produce a mixture. 20 mg/mL NHS-activated acridinium ester was then mixed with the mixture at a ratio of 8 (μL):1 (mg) to the anti-human myoglobin monoclonal antibody for a reaction at 4° C. under shaking for 4 hours. The resulting product was then dialyzed against PBS (pH 7.4) overnight to remove free acridinium ester. The dialyzed product was preserved at 4° C. for use.

Fluorescent microsphere labeling:

0.5 mL of the microsphere was cleaned with 0.1 M phosphate buffer (pH 7.2) and centrifuged at 13,000 rpm. Such process was repeated three times. The resulting microsphere was redissolved with the 0.1 M phosphate buffer (pH 7.2) to 1 mL to produce a blend. The blend was then added with 200 μg of anti-human myoglobin monoclonal antibody and mixed uniformly. Other procedures were referred to Experimental 5.

Preparation of fluorescent microsphere-labeled absorption membrane: referring to Experiment 5.

Preparation of polyclonal antibody-imprinted membrane: referring to Experiment 5.

Method for assembling semi-finished product: referring to Experiment 5.

Test group:

The test strip prepared above was placed in a centrifugation rotor with its fluorescent microsphere-labeled absorption membrane facing upwards. The test stripe was added with 80 μL of each of the prepared human myoglobin solutions of different concentrations on its fluorescent microsphere-labeled absorption membrane followed by a standing for 2 minutes. The test strip was then centrifuged at 1,000 rpm for 1 minute, and was added with 80 μL of acridinium ester-labeled goat anti-mouse IgG polyclonal antibody on its fluorescent microsphere-labeled absorption membrane followed by a standing for 2 minutes. The test strip was then centrifuged at 1,000 rpm for 1 minute, and was further added with 80 μL of 50 mM PBS (pH 7.4) containing 0.05% Tween-20 on its fluorescent microsphere-labeled absorption membrane followed by a centrifugation at 2,000 rpm for 30 seconds for cleaning. The cleaning was repeated once. The test strip was then to obtain a polyclonal antibody-imprinted membrane detection line, which was placed in a transparent test tube. The test tube was then placed in a chemiluminescence detector and added with a luminescence-activating reagent with a delay time set to 0.30 seconds and a luminescence integration time set to 10 seconds. The experiment was repeated three times, and the results were averaged and used to plot a concentration-luminescence curve and calculate a correlation coefficient thereof.

Control group:

The test strip prepared above was placed on a table, and was added with 80 μL of each of the prepared human myoglobin solutions of different concentrations on its fluorescent microsphere-labeled adsorption membrane followed by a standing. After the liquid on the fluorescent microsphere-labeled adsorption membrane flowed completely into the nitrocellulose membrane via chromatography, the fluorescent microsphere-labeled adsorption membrane was added with 80 μL of acridinium ester-labeled goat anti-mouse IgG polyclonal antibody followed by a standing to allow the liquid flow completely into the nitrocellulose membrane. The fluorescent microsphere-labeled adsorption membrane was further added with 80 μL of 50 mM PBS (pH 7.4) containing 0.05% Tween-20 followed by a standing to allow the liquid flow completely into the nitrocellulose membrane for cleaning. The cleaning was repeated once. The test strip was then cut to obtain a polyclonal antibody-imprinted membrane detection line, which was placed in a transparent test tube. The test tube was then placed in the chemiluminescence detector and added with the luminescence-activating reagent with a delay time set to 0.30 seconds and a luminescence integration time set to 10 seconds. The experiment was repeated three times, and the results were averaged and used to plot a concentration-luminescence curve and calculate a correlation coefficient thereof.

3. Experimental Results

In the present experiment, the fluorescent microsphere was used as a liquid phase reaction carrier, and method F was used to perform the experiment. As a result, the average detection processing time of the test strip of the test group of the present invention was 7.8 minutes, while that of the control group (without centrifugation processing) was 59 minutes. The test data of the test group of the present invention showed a good correlation between concentration and luminescence value with a correlation coefficient r of 0.990. While the results of the control group were below 100 ng/mL and substantially no correlation between concentration and luminescence value was found with a correlation coefficient r of 0.912, P<0.05, which was mainly resulted from an undesirable cleaning effect of the chemiluminescent enzyme due to a non-specific binding to the solid phase membrane, thereby resulting in a high background. The present invention has significantly better test results than those obtained using the assay without centrifugation processing, which indicated that the linearity and accuracy of the detection were improved using the technique of the present invention improves when compared to the prior art. The experimental results are shown in Table. 6.

TABLE 6
Experimental results (luminescence value) of the paper-chromatography
and chemiluminescence assay using method F

| | Concentration (ng/mL) | | | | | |
|---|---|---|---|---|---|---|
| | 0.1 | 1.0 | 10 | 100 | 1000 | r value |
| Test group | 52314 | 132451 | 352768 | 2133456 | 10298756 | 0.990 |
| Control group | 1198716 | 1211096 | 1354231 | 5438765 | 10786543 | 0.912 |

Experiment 7: Effect of Centrifugal Speed on Test Results 1. Experimental Materials:

Referring to Experiment 1.

2. Experimental Methods:

Control group was not required. The human myoglobin solution in the test group was sampled respectively at a centrifugal speed of 500, 1,000, 2,000, 3,000, 4,000 and 5,000 rpm. The cleaning was performed using a centrifugal speed of 2,000 rpm. Other procedures were referred to Experiment 1.

3. Experimental Result

In the present experiment, the colloidal gold micro particle was used as a liquid phase reaction carrier, and method A was used to perform the experiment. Effects of different centrifugal speeds on the test results were investigated. Human myoglobin samples of different concentrations were tested in the experiment. The experimental results are shown in Table 7. As shown in Table 7, the detection accuracy was related to the centrifugal speed. A desirable result was obtained with a centrifugal speed of 500, 1,000 and 2,000 rpm, respectively. While the separately use of a centrifugal speed of 3,000, 4,000 and 5,000 rpm may produce a significant decrease in luminescence value, and an effect on the detection sensitivity and accuracy. Such results indicated that an optimal speed for detecting myoglobin in the present invention should be below 2,000 rpm.

TABLE 7
Effect of centrifugal speed on the test results (luminescence value)

| Centrifugal speed | Concentration (ng/mL) | | | | | |
|---|---|---|---|---|---|---|
| (rpm) | 0.1 | 1.0 | 10 | 100 | 1000 | r value |
| 500 | 12587 | 49716 | 132763 | 685546 | 4213450 | 0.994 |
| 1000 | 13215 | 52519 | 140587 | 1211394 | 3988763 | 0.994 |
| 2000 | 10352 | 51590 | 151777 | 612041 | 3982240 | 0.996 |
| 3000 | 11298 | 32173 | 127821 | 421367 | 3623551 | 0.991 |
| 4000 | 12308 | 39871 | 112534 | 321986 | 3345242 | 0.998 |
| 5000 | 11235 | 32152 | 54321 | 213321 | 2687251 | 0.962 |

### EXAMPLE 2

### Preparation of Combined Kit of Fluorescence Immune Chromatography Assay with Centrifuge Isolation

The combination kit of fluorescence immune chromatography assay with centrifuge isolation of the present invention includes an analyte-specific binding substance, a micro particle, a solid phase membrane, a fluorescent substance, a centrifugation device, a fluorescence detector, a fluorescence excitation structure, and a second analyte-specific binding substance or an intermediate B, a labeled adsorption membrane pad, a water-absorbing membrane pad, and a support base plate.

The following experiments are used to illustrate the detection method of the present invention and effects thereof, but are not intended to limit the scope of the present invention. Experimental methods used in the experiments described below are conventional methods unless specified otherwise. Materials, reagents and the like used in the following experiments are commercially available unless specified otherwise.

Experiment 1 for Fluorescence Immune Chromatography Assay with Centrifuge Isolation

Colloidal gold particle was used as a liquid phase reaction carrier, and method A was used to perform the experiment.

1. Experimental Materials

Nitrocellulose membrane (MILLIPORE), polyester cellulose membrane (Alstrom, USA), chloroauric acid (SIGMA), fluorescence quantitative analyzer (Shanghai Jinguo Biotech Company HG-98), fluorescein isothiocyanate (FITC, SIGMA), and other materials were referred to Experiment 1 of Example 1.

In the present experiment, the anti-human myoglobin polyclonal antibody was used as a second analyte-specific binding substance; the anti-human myoglobin monoclonal antibody was used as an analyte-specific binding substance; fluorescein isothiocyanate was used as a fluorescent substance; human myoglobin was used as an analyte; the desk centrifuge was used as a centrifugation device; the nitrocellulose membrane was used as a solid phase membrane; the chloroauric acid was used as a material for preparing a micro particle; and the fluorescence quantitative analyzer was used as a fluorescence detector.

2. Experimental Methods

Preparation of human myoglobin solutions: referring to Experiment 1 of Example 1.

FITC labeling of anti-human myoglobin monoclonal antibody:

The Marsshall method was adopted. 3 mg/mL anti-human myoglobin monoclonal antibody solution was added with 0.5 M (pH 9.0) carbonate buffer at a volume ratio of 10:1 to produce a mixture. The mixture was stirred electromagnetically for 5 minutes. A 1 mg/mL FITC solution was prepared with 50 mM phosphate (PBS) buffer (pH 8.0), and was slowly added to the mixture under stirring at an amount of 30 μL/mL. The reaction was then stirred at 4° C. in the dark for 12 hours to produce a labeled antibody solution. The labeled antibody solution was centrifuged at 2,500 rpm for 20 minutes. The precipitate was removed and the supernatant was then transferred to a dialysis bag, and dialyzed overnight against 50 mM PBS at 4° C. to produce a marker. The marker was passed through a Sephadex gel G-25 column to isolate the labeled fluorescent antibody from free FITC, i.e., FITC-labeled anti-human myoglobin monoclonal antibody. The FITC-labeled anti-human myoglobin monoclonal antibody was collected and preserved at −20° C. in the dark for use.

Colloidal gold micro particle labeling:

100 μg of the FITC-labeled anti-human myoglobin monoclonal antibody was quickly added to a colloidal gold micro particle solution. Other procedures were referred to Experiment 1 of Example 1.

Preparation of colloidal gold particle-labeled absorption membrane: referring to Experiment 1 of Example 1.

Preparation of polyclonal antibody-imprinted membrane: referring to Experiment 1 of Example 1.

Method for assembling semi-finished product: referring to Experiment 1 of Example 1.

Test group: Procedures were referred to Experiment 1 of Example 1. The test strip was placed on a fluorescence analyzer for reading a fluorescence value. The experiment was repeated three times, and the results were averaged and used to plot a concentration-luminescence curve and calculate a correlation coefficient thereof.

Control group:

Procedures were referred to Experiment 1 of Example 1. The test strip was placed on the fluorescence analyzer for reading the fluorescence value. The experiment was repeated three times, and the results were averaged and used to plot a concentration-luminescence curve and calculate a correlation coefficient thereof.

3. Experimental Results

In the present experiment, the colloidal gold micro particle was used as a liquid phase reaction carrier, and method A was used to perform the experiment. As a result, the average detection processing time of the test strip of the test group of the present invention was 3.5 minutes, while that of the control group (without centrifugation processing) was 38 minutes. The test data of the test group of the present invention showed a good correlation between concentration and luminescence value with a correlation coefficient r of 0.989. While an undesirable correlation between concentration and luminescence value of the control group was obtained with a concentration below 10 ng/mL. The ratio of the test data obtained at 10 ng/mL to that obtained at 1 ng/mL of the test group of the invention was 7.31, while that of the control group was only 1.50, which was mainly resulted from an undesirable cleaning effect of the fluorescent substance due to a non-specific binding to the solid phase membrane, thereby resulting in a high background. The experimental results indicated that the technique of the present invention can not only reduce the detection time, but also improve the linearity and accuracy of the detection when compared with the prior art. The experimental results were shown in Table 15.

TABLE 15
Experimental results (luminescence value) of the fluorescence immune
chromatography assay with centrifuge isolation using method A

| | Concentration (ng/mL) | | | | | |
|---|---|---|---|---|---|---|
| | 1.0 | 10 | 100 | 500 | 1000 | r value |
| Test group | 2916 | 21323 | 131618 | 212014 | 368224 | 0.989 |
| Control group | 23512 | 35235 | 143521 | 254231 | 381670 | 0.984 |

Experiment 4 for Fluorescence Immune Chromatography Assay with Centrifuge Isolation

Colloidal gold particle was used as a liquid phase reaction carrier, and method B was used to perform the experiment.

1. Experimental Materials

FITC-labeled goat anti-mouse IgG polyclonal antibody (Shanghai Yiyan Biotech); and other materials were referred to Experiment 1.

In the present experiment, the FITC-labeled goat anti-mouse IgG polyclonal antibody was used as a fluorescein-labeled specific conjugate of an analyte-specific binding substance, i.e., a fluorescent substance marker. Others were referred to Experiment 1.

2. Experimental Methods

Preparation of human myoglobin solutions: referring to Experiment 1.

Colloidal gold particle labeling

10 mL of colloidal gold solution was adjusted to pH 8.3 with 10% potassium carbonate to produce a mixture. The mixture was then quickly added with 100 μg of anti-human myoglobin monoclonal antibody to a final concentration of 10 μg/mL.

The reaction was shaken and mixed uniformly. Other procedures were referred to Experiment 1.

Preparation of colloidal gold particle-labeled absorption membrane: referring to Experiment 1.

Preparation of polyclonal antibody-imprinted membrane: referring to Experiment 1.

Method for assembling semi-finished product: referring to Experiment 1.

Test group:

The test strip prepared above was placed in a centrifugation rotor with its colloidal gold particle-labeled adsorption membrane facing upwards. The test strip was added with 80 μL of each of the prepared human myoglobin solutions of different concentrations on its colloidal gold particle-labeled adsorption membrane followed by a standing for 2 minutes. The test strip was then centrifuged at 1,000 rpm for 1 minute, and was added with 80 μL of the FITC-labeled goat anti-mouse IgG polyclonal antibody solution on its colloidal gold particle-labeled adsorption membrane followed by a standing for 2 minutes. The test strip was then centrifuged at 1,000 rpm for 1 minute, and was further added with 80 μL of 50 mM PBS (pH 7.4) containing 0.05% Tween-20 on its colloidal gold particle-labeled adsorption membrane. The test strip was then centrifuged at 2,000 rpm for 30 seconds for cleaning. The test strip was transferred to the fluorescence analyzer for a reading of the fluorescence value. The experiment was repeated three times, and the results were averaged and used to plot a concentration-luminescence curve and calculate a correlation coefficient thereof.

Control group:

The test strip prepared above was placed on a table, and was added with 80 μL of each of the prepared human myoglobin solutions of different concentrations on its colloidal gold particle-labeled adsorption membrane followed by a standing. After the red-color colloidal gold micro particle marker on the colloidal gold micro particle-labeled adsorption membrane flowed completely into the nitrocellulose membrane via chromatography, the colloidal gold particle-labeled adsorption membrane was added with 80 μL of FITC-labeled goat anti-mouse IgG polyclonal antibody solution followed by a standing to allow the liquid flow completely into the nitrocellulose membrane. The colloidal gold particle-labeled adsorption membrane was further added with 80 μL of 50 mM PBS (pH 7.4) containing 0.05% Tween-20 followed by a standing to allow the liquid to flow completely into the nitrocellulose membrane. The test strip was then placed on the fluorescence analyzer for a reading of the fluorescence value. The experiment was repeated three times, and the results were averaged and used to plot a concentration-luminescence curve and calculate a correlation coefficient thereof.

3. Experimental Results

In the present experiment, the colloidal gold micro particle was used as a liquid phase reaction carrier, and method B was used to perform the experiment. As a result, the average detection processing time of the test strip of the test group of the present invention was 6.8 minutes, while that of the control group (without centrifugation processing) was 51 minutes. The test data of the test group of the present invention showed a good correlation between concentration and luminescence value with a correlation coefficient r of 0.989. While a poor correlation between concentration and luminescence value of the control group was obtained with a concentration below 10 ng/mL. The ratio of the test data obtained at 10 ng/mL to that obtained at 1 ng/mL of the test group of the present invention was 9.1, while that of the control group was only 1.5, which was mainly resulted from an undesirable cleaning effect of the fluorescent substance due to a non-specific binding to the solid phase membrane, thereby resulting in a high background. The experimental results indicated that the technique of the present invention can not only reduce the detection time, but also improve the linearity and accuracy of the detection when compared with the prior art.

The experimental results are shown in Table 18.

TABLE 18
Experimental results (luminescence value) of the fluorescence immune
chromatography assay with centrifuge isolation using method B

| | Concentration (ng/mL) | | | | | |
|---|---|---|---|---|---|---|
| | 1 | 10 | 100 | 500 | 1000 | r value |
| Test group | 5136 | 46527 | 156325 | 462678 | 531268 | 0.989 |
| Control group | 42567 | 62123 | 176523 | 452341 | 532806 | 0.981 |

Experiment 5 for Fluorescence Immune Chromatography Assay with Centrifuge Isolation

Fluorescent microsphere was used as a liquid phase reaction carrier, and method C was used to preform the experiment.

1. Experimental Materials

NHS-activated biotin (SIGMA), avidin (SIGMA), and other materials were referred to Experiment 2.

In the present experiment, the biotin was used as an intermediate A; and the avidin was used as an intermediate B. Others were referred to Experiment 2.

2. Experimental Methods

Preparation of human myoglobin solutions: referring to Experiment 1.

Biotin-labeling Anti-human myoglobin polyclonal antibody:

Anti-human myoglobin polyclonal antibody was dialyzed against 0.1 M pH 9.5 sodium carbonate buffer overnight and adjusted to a final concentration of 2 mg/mL to produce an antibody solution. 20 mg of NHS-activated biotin was dissolved in 1 mL of dimethylformamide to produce a biotin solution. 50 μL of the biotin solution was added to the antibody solution. The reaction was reacted at room temperature for 4 hours. The resulting product was dialyzed overnight against PBS buffer and preserved at −20° C.

Fluorescent microsphere labeling: referring to Experiment 2.

Preparation of fluorescent microsphere-labeled absorption membrane:

The fluorescent microsphere-labeled antibody solution was diluted with colloidal buffer (1% BSA, 3% sucrose, 50 mM PBS, pH 7.4) to an OD₅₃₀ of 30. The diluted solution was then added with the biotin-labeled anti-human myoglobin polyclonal antibody at an amount of 10 μg/mL and mixed uniformly. Other procedures were referred to Experiment 1.

Preparation of avidin-imprinted membrane:

The avidin solution was diluted to a concentration of 1 mg/mL with 50 mM phosphate buffer (pH 7.4). The imprinter was started and loaded with the diluted avidin solution. Other procedures were referred to Experiment 1.

Method for assembling semi-finished product:

The dehumidifier was started to reduce the humidity in the operating room to less than 25%. The water-absorbing paper membrane pad and the colloidal gold micro particle-labeled adsorption membrane were pasted respectively on each end of the avidin-imprinted membrane. Other procedures were referred to Experiment 1.

Test group:

The test strip prepared above was placed in a centrifugation rotor with its fluorescent microsphere-labeled absorption membrane facing upwards. The test stripe was added with 80 μL of each of the prepared human myoglobin solutions of different concentrations on its fluorescent microsphere-labeled absorption membrane followed by a standing for 2 minutes. The test strip was then centrifuged at 1,000 rpm for 1 minute, and was added with 80 μL of 50 mM PBS (pH 7.4) containing 0.05% Tween-20 on its fluorescent microsphere-labeled absorption membrane. The test strip was then centrifuged at 2,000 rpm for 30 seconds for a cleaning. The test strip was then transferred to the fluorescent analyzer for a reading of the fluorescence value. The experiment was repeated three times, and the results were averaged and used to plot a concentration-luminescence curve and calculate a correlation coefficient.

Control group:

The test strip prepared above was placed on a table, and was added with 80 μL of each of the prepared human myoglobin solutions of different concentrations on its fluorescent microsphere-labeled adsorption membrane followed by a standing. After the liquid on the fluorescent microsphere-labeled adsorption membrane flowed completely into the nitrocellulose membrane via chromatography, the fluorescent microsphere-labeled adsorption membrane was added with 80 μL of 50 mM PBS (pH 7.4) containing 0.05% Tween-20 followed by a standing to allow the liquid flow completely into the nitrocellulose membrane for cleaning. The test strip was then transferred to the fluorescent analyzer for a reading of the fluorescence value. The experiment was repeated three times, and the results were averaged and used to plot a concentration-luminescence curve and calculated a correlation coefficient thereof.

3. Experimental Results

In the present experiment, the fluorescent microsphere was used as a liquid phase reaction carrier, and method C was used to perform the experiment. As a result, the average detection processing time of the test strip of the test group of the present invention was 3.8 minutes, while that of the control group (without centrifugation processing) was 37 minutes. The test data of the test group of the present invention showed a good correlation between concentration and luminescence value with a correlation coefficient r of 0.989. While a poor correlation between concentration and luminescence value of the control group was obtained with a concentration below 10 ng/mL. The ratio of the test data obtained at 10 ng/mL to that obtained at 1 ng/mL of the test group of the present invention was 5.0, while that of the control group was only 2.4, which was mainly resulted from an undesirable cleaning effect of the fluorescent substance due to a non-specific binding to the solid phase membrane, thereby resulting in a high background. The experimental results indicated that the technique of the present invention can not only reduce the detection time, but also improve the linearity and accuracy of the detection when compared to the prior art. The experimental results were shown in Table 19.

TABLE 19
Experimental results (luminescence value) of the fluorescence immune
chromatography assay with centrifuge isolation using method C

| | Concentration (ng/mL) | | | | | |
|---|---|---|---|---|---|---|
| | 1 | 10 | 100 | 500 | 1000 | r value |
| Test group | 15163 | 77256 | 193215 | 362783 | 461567 | 0.989 |
| Control group | 32156 | 78569 | 201347 | 453309 | 523210 | 0.999 |

Experiment 6 For Fluorescence Immune Chromatography Assay with Centrifuge Isolation

Fluorescent microsphere was used as a liquid phase reaction carrier, and method D was used to perform the experiment.

1. Experimental Materials

NHS-activated biotin (SIGMA), avidin (SIGMA), FITC-labeled goat anti-mouse IgG polyclonal antibody (Shanghai Yiyan Biotech), and other materials were referred to Experiment 2.

In the present experiment, the biotin was used as an intermediate A; the avidin was used as an intermediate B; and the FITC-labeled goat anti-mouse IgG polyclonal antibody was used as a fluorescin-labeled specific conjugate of an analyte-specific binding substance, i.e., a fluorescent substance marker. Others were referred to Experiment 2.

Preparation of human myoglobin solutions: referring to Experiment 1.

Biotin labeling of anti-human myoglobin polyclonal antibody: referring to Experiment 4.

Fluorescent microsphere labeling: referring to Experiment 2.

Preparation of fluorescent microsphere-labeled absorption membrane:

The colloidal gold particle-labeled antibody solution was diluted with a colloidal buffer (1% BSA, 3% sucrose, 50 mM PBS, pH 7.4) to an OD₅₃₀ of 30. The diluted solution was then added with the biotin-labeled anti-human myoglobin polyclonal antibody at an amount of 10 μg/mL, and was mixed uniformly. Other procedures were referred to Experiment 2.

Preparation of avidin-imprinted membrane:

The avidin solution was diluted to a concentration of 1 mg/mL with 50 mM phosphate buffer (pH 7.4). The imprinter was started and loaded with the diluted avidin solution. Other procedures were referred to Experiment 2.

Method for assembling semi-finished product:

The dehumidifier was started to reduce the humidity in the operating room to less than 25%. The water-absorbing paper membrane pad and the fluorescent microsphere-labeled adsorption membrane were pasted respectively on each end of the avidin-imprinted membrane. Other procedures were referred to Experiment 2.

Test group:

The test strip prepared above was placed in a centrifugation rotor with its fluorescent microsphere-labeled adsorption membrane facing upwards. The test strip was added with 80 μL of each of the prepared human myoglobin solutions of different concentrations on its fluorescent microsphere-labeled adsorption membrane followed by a standing for 2 minutes. The test strip was then centrifuged at 1,000 rpm for 1 minute, and was added with 80 μL of the FITC-labeled goat anti-mouse IgG polyclonal antibody solution on its fluorescent microsphere-labeled adsorption membrane followed by a standing for 2 minutes. The test strip was then centrifuged at 1,000 rpm for 1 minute, and was further added with 80 μL of 50 mM PBS (pH 7.4) containing 0.05% Tween-20 on its colloidal gold particle-labeled adsorption membrane. The test strip was then centrifuged at 2,000 rpm for 30 seconds for cleaning. The test strip was transferred to the fluorescence analyzer for a reading of the fluorescence value. The experiment was repeated three times, and the results were averaged and used to plot a concentration-luminescence curve and calculate a correlation coefficient thereof.

Control group:

The test strip prepared above was placed on a table, and was added with 80 μL of each of the prepared human myoglobin solutions of different concentrations on its fluorescent microsphere-labeled adsorption membrane followed by a standing. After the liquid on the fluorescent microsphere-labeled adsorption membrane flowed completely into the nitrocellulose membrane, the fluorescent microsphere-labeled adsorption membrane was added with 80 μL of FITC-labeled goat anti-mouse IgG polyclonal antibody solution followed by a standing to allow the liquid flow completely into the nitrocellulose membrane. The fluorescent microsphere-labeled adsorption membrane was further added with 80 μL of 50 mM PBS (pH 7.4) containing 0.05% Tween-20 followed by a standing to allow the liquid flow completely into the nitrocellulose membrane. The test strip was transferred to the fluorescence analyzer for a reading of the fluorescence value. The experiment was repeated three times, and the results were averaged and used to plot a concentration-luminescence curve and calculate a correlation coefficient thereof.

3. Experimental Results

In the present experiment, the fluorescent microsphere was used as a liquid phase reaction carrier, and method D was used to perform the experiment. As a result, the average detection processing time of the test strip of the test group of the present invention was 7.1 minutes, while that of the control group (without centrifugation processing) was 52 minutes. The test data of the test group of the present invention showed a good correlation between concentration and luminescence value with a correlation coefficient r of 0.978. While an undesirable correlation between concentration and luminescence value of the control group was obtained for a concentration below 10 ng/mL. The ratio of the test data obtained at 10 ng/mL to that obtained at 1 ng/mL of the test group of the present invention was 14.3, while that of the control group was only 1.8, which was mainly resulted from an undesirable cleaning effect of the fluorescent substance due to a non-specific binding to the solid phase membrane, thereby resulting in a high background. The experimental results indicated that the technique of the present invention can not only reduce the detection time, but also improve the linearity and accuracy of the detection when compared with the prior art. The experimental results were shown in Table 20.

TABLE 20
Experimental results (luminescence value) of the fluorescence immune
chromatography assay with centrifuge isolation using method D

| | Concentration (ng/mL) | | | | | |
|---|---|---|---|---|---|---|
| | 1 | 10 | 100 | 500 | 1000 | r value |
| Test group | 2967 | 42317 | 191021 | 415322 | 487652 | 0.978 |
| Control group | 43218 | 79843 | 198627 | 423785 | 505412 | 0.995 |

Experiment 7: Effect of Centrifugal Speed on Test Results

Fluorescent microsphere was used as a liquid phase reaction carrier, and method A was used to perform the experiment.

1. Experimental Materials: Referring to Experiment 2.

2. Experimental Methods:

Control group was not required. The test group was respectively sampled at a centrifugal speed of 500, 1,000, 2,000, 3,000, 4,000 and 5,000 rpm. The cleaning was performed at a centrifugal speed of 2,000 rpm. The centrifugation was performed after the samples were added followed by a standing for 1 minute. Other procedures were referred to Experiment 2.

3. Experimental Results:

In the present experiment, the fluorescent microsphere was used as a liquid phase reaction carrier, and method A was used to perform the experiment. Effects of different centrifugal speeds on the test results were investigated. Human myoglobin samples of different concentrations were tested in the experiment, and the experimental results were shown in Table 21. As shown in Table 21, the detection accuracy was related to the centrifugal speed. The test results obtained using centrifugal speeds of 500, 1,000 and 2,000 rpm met the requirement, while the test results obtained using centrifugal speeds of 3,000, 4,000 and 5,000 rpm indicated a significant decrease in fluorescence value, which affected the detection sensitivity and accuracy. Such results demonstrated that an optimal speed for detecting myoglobin in the present invention should be below 2,000 rpm.

TABLE 21
Effect of centrifugal speed on the test results (luminescence value)

| Centrifugal speed | Concentration (ng/mL) | | | | | |
|---|---|---|---|---|---|---|
| (rpm) | 1 | 10 | 100 | 500 | 1000 | r value |
| 500 | 1587 | 25245 | 123211 | 267652 | 362908 | 0.981 |
| 1000 | 2645 | 31021 | 145672 | 287601 | 421350 | 0.984 |
| 2000 | 1987 | 28762 | 123269 | 301233 | 412356 | 0.984 |
| 3000 | 1109 | 2133 | 58901 | 122571 | 296578 | 0.975 |
| 4000 | 1078 | 2376 | 41234 | 87653 | 267546 | 0.999 |
| 5000 | 1198 | 1987 | 31005 | 78654 | 257550 | 0.973 |

Experiment 8 for Effects of Particle Size of Micro Particle on Test Results

Polystyrene microsphere was used as a liquid phase reaction carrier, and method A was used to perform the experiment.

1. Experimental Materials

Carboxylated polystyrene microspheres (particle sizes of 35, 130, 376, 600, 1000 nm, Shanghai Taoyu International), and other materials were referred to Experiment 1.

In the present experiment, the polystyrene microsphere was a micro particle. Others were referred to Experiment 1.

2. Experimental Methods

Preparation of human myoglobin solutions: referring to Experiment 1.

FITC labeling of anti-human myoglobin monoclonal antibody: referring to Experiment 1.

Polystyrene microsphere labeling

2 mL of 0.1 M MES solution (pH 5.0), 2 mg of FITC-labeled anti-human myoglobin monoclonal antibody, 0.2 mL of 5% (w/v) microspheres and 20 mg of EDC were mixed uniformly to produce a mixture. The mixture was then added with 10 mg of NHS. The reaction was shaken with a rotating shaker (oscillator) for 2 hours at room temperature. The resulting product was then centrifuged at 3000×g for 15 minutes. The supernatant was discarded and the obtained precipitate was added with 2 mL of blocking buffer containing 20 mM PBS and 1% BSA for blocking at room temperature for 1 hour. The blocked product was resuspended with 4 mL of 50 mM PBS (pH 7.4) for cleaning, and the cleaning was repeated once to produce a polystyrene microsphere-labeled antibody. The polystyrene microsphere-labeled antibody was added with 2 mL of 50 mM PBS and preserved at 4° C. for use.

Preparation of polystyrene microsphere-labeled absorption membrane

The polystyrene microsphere-labeled antibody solution was diluted with 50 mM PBS (pH 7.4) to a microsphere content of 1% (w/v). The imprinter was started and loaded with the antibody solution, and the pressurized nitrogen was turned on. The polyester cellulose membrane was used to perform membrane imprinting with the imprinting conditions set as follows: a moving speed of airbrush was 30 mm/sec, and a liquid propelling speed was 5.0 μL/cm. The imprinted membrane was dried at 37° C. in a drying oven for 6 hours, and was then preserved in a sealed bag containing desiccant for later use.

Preparation of polyclonal antibody-imprinted membrane: referring to Experiment 1.

Method for assembling semi-finished product: referring to Experiment 1.

Test group:

The test strip prepared above was placed in a centrifugation rotor with its polystyrene microsphere-labeled adsorption membrane facing upwards. The test strip was added with 80 μL of each of the prepared human myoglobin solutions of different concentrations on its polystyrene microsphere-labeled adsorption membrane followed by a standing for 2 minutes. The test strip was then centrifuged at 1,000 rpm for 1 minute, and was added with 80 μL of 50 mM PBS (pH 7.4) containing 0.05% Tween-20 on its polystyrene microsphere-labeled adsorption membrane. The test strip was then centrifuged at 2,000 rpm for 30 seconds for cleaning. The test strip was transferred to the fluorescence analyzer for a reading of the fluorescence value. The experiment was repeated three times, and the results were averaged and used to plot a concentration-luminescence curve and calculate a correlation coefficient thereof.

3. Experimental Results

In the present experiment, the polystyrene microsphere was used as a liquid phase reaction carrier, and method A was used to perform the experiment. Effects of different particle sizes on the test results were investigated. The results showed that the test data showed a good correlation between concentration and luminescence with a correlation coefficient r>0.98 with the use of particle diameters of 35, 130, 376, 600 and 1,000 nm. This demonstrated that micro particles of different sizes are applicable for the present invention. The experimental results were shown in Table 22.

TABLE 22
Effect of particle size of the micro particle
on the test results (luminescence value)

| Particle size | Concentration (ng/mL) | | | | | |
|---|---|---|---|---|---|---|
| (nm) | 1 | 10 | 100 | 500 | 1000 | r value |
| 35 | 2316 | 26751 | 145290 | 302962 | 450012 | 0.987 |
| 130 | 1982 | 21338 | 142941 | 282769 | 394864 | 0.987 |
| 376 | 2901 | 31890 | 149782 | 290031 | 423365 | 0.985 |
| 600 | 3124 | 19874 | 150028 | 321248 | 432105 | 0.993 |
| 1000 | 2317 | 26532 | 154392 | 321876 | 432105 | 0.986 |

Comparative Experiment 9: Time-Associated Detection Stability Between the Present Invention and the Prior Art

Fluorescent microsphere was used as a liquid phase reaction carrier, and method A was used to perform the experiment.

1. Experimental Materials

Referring to Experiment 2.

2. Experimental Methods

Preparation of human myoglobin solutions

A human myoglobin solution of a known concentration was diluted to series of human myoglobin solutions of 100 ng/mL with a sample dilution buffer (1% BSA, 100 mM glycine, 50 mM PBS, 150 mM NaCl, pH 7.4).

The treatment of the test strip was referred to Experiment 2. After the cleaning, the test strip was placed on the fluorescence analyzer for a reading of the fluorescence value, wherein a reading was performed every 2 minutes and performed successively 5 times. The experiment was repeated 3 times, and the results were averaged.

3. Experimental Results

In the present experiment, the fluorescent microsphere was used as a liquid phase reaction carrier, and method A was used to perform the experiment. The stability of the fluorescence detection value with the extension of the standing time after the cleaning was investigated, and the experimental results were shown in Table 23. The test data of the test group of the present invention was substantially stable during the subsequent 8-minute continuous observation period, while the test data of the control group obtained using the prior art increased over time during the subsequent 8-minute continuous observation period. The results indicated that the technique of the present invention can improve the stability of detection.

TABLE 23
Results (luminescence value) of the comparative experiment
of time-associated detection stability between
the present invention and the prior art

| | Delay detection time (min) | | | | |
|---|---|---|---|---|---|
| | 0 | 2 | 4 | 6 | 8 |
| The present invention | 145321 | 150987 | 149125 | 145679 | 152876 |
| The prior art | 152314 | 167891 | 178652 | 190062 | 192136 |

### EXAMPLE 3

### Preparation of Combined Kit of Colloidal Gold Immune Chromatography Assay with Centrifuge Isolation

The combined kit of immune chromatography colloidal gold assay with centrifuge isolation includes an analyte-specific binding substance, a colloidal gold micro particle, a solid phase membrane, a centrifugation device, a colloidal gold detector, and a second analyte-specific binding substance or an intermediate B, a labeled absorption membrane pad, a water-absorbing membrane pad and a support base plate.

The following experiments were used to illustrate the detection method of the present invention and effects thereof, but are not intended to limit the scope of the present invention. Experimental methods used in the experiments described below are conventional methods unless specified otherwise. Materials, reagents and the like used in the following experiments are commercially available unless specified otherwise.

Experiment 1 for Colloidal Gold Immune Chromatography Assay with Centrifuge Isolation of the Present Invention

Colloidal gold micro particle was used as a liquid phase reaction carrier, and method A was used to perform the experiment.

1. Experimental Materials

Nitrocellulose membrane (MILLIPORE), polyester cellulose membrane (Alstrom, USA), water-absorbing paper membrane pad (S&S, USA), chloroauric acid (SIGMA), colloidal gold quantitative chromatography analyzer (Skannex, Norway), and other materials were referred to Experiment 1 of Example 1.

In the present experiment, the anti-human myoglobin polyclonal antibody was used as a second analyte-specific binding substance; the anti-human myoglobin monoclonal antibody was used as an analyte-specific binding substance; the human myoglobin was used as the analyte; the desk centrifuge was used as a centrifugation device; the nitrocellulose membrane was used as a solid phase membrane; the chloroauric acid was used as a material for preparing a colloidal gold micro particle; and the colloidal gold quantitative chromatography analyzer was used as a colloidal gold detector.

2. Experimental Methods

Preparation of human myoglobin solutions

A human myoglobin solution of a known concentration was diluted to series of human myoglobin solutions of 3, 10, 30, 70, 100, 150 and 200 ng/mL with a sample dilution buffer (1% BSA, 100 mM glycine, 50 mM PBS, 150 mM NaCl, pH 7.4).

Colloidal gold micro particle labeling: referring to Experiment 1 of Example 1. Preparation of colloidal gold micro particle-labeled membrane: referring to Experiment 1 of Example 1.

Preparation of polyclonal antibody-imprinted membrane: referring to Experiment 1 of Example 1.

Method for assembling semi-finished product: referring to Experiment 1 of Example 1.

Test group:

Procedures were referred to Experiment 1 of Example 1. The test strip was transferred to the colloidal gold quantitative chromatography analyzer for a reading of the chroma value. The experiment was repeated three times, and the results were averaged and used to plot a concentration-chroma value curve and calculate a correlation coefficient thereof.

Control group:

Procedures were referred to Experiment 1 of Example 1. After being taken out, the test strip was placed on the colloidal gold quantitative chromatography analyzer for a reading of the chroma value. The experiment was repeated three times, and the results were averaged and used to plot a concentration-chroma value curve and calculate a correlation coefficient thereof.

3. Experimental Results

In the present experiment, the colloidal gold micro particle was used as a liquid phase reaction carrier, and method A was used to perform the experiment. As a result, the average detection processing time of the test strip of the test group of the present invention was 3.6 minutes, while that of the control group (without centrifugation processing) was 37 minutes. The test data of the test group of the present invention showed a good correlation between concentration and chroma value, and a correlation coefficient r of 0.989 was obtained when the upper detection limit was set to 150 ng/mL and the linear detection range was 3-150 ng/mL. As for the control group, a correlation coefficient r of 0.992 was obtained, which met the detection requirement of r>0.98 when the upper detection limit was set to 100 ng/mL, and linear detection range was 3-100 ng/mL. The ratio of the test data obtained at 10 ng/mL to that obtained at 3 ng/mL of the test group of the present invention was 2.26, while that of the control group was only 1.09. The above differences were mainly resulted from an undesirable cleaning effect of the colloidal gold micro particle due to a non-specific binding to the solid phase membrane, thereby resulting in a high background. The experimental results indicated that the technique of the present invention can not only reduce the detection time, but also improve the linearity and accuracy of the detection when compared with the prior art. The experimental results were shown in Table 26.

TABLE 26
Experimental results (chroma value) of the immune chromatography
colloidal gold assay with centrifuge isolation using method A

| | Concentration (ng/mL) | | | | | | | |
|---|---|---|---|---|---|---|---|---|
| | 3 | 10 | 30 | 70 | 100 | 150 | 200 | r value |
| Test group | 2.3 | 5.2 | 25.7 | 67.8 | 108.9 | 132.1 | 136.8 | 0.963 |
| Control group | 5.3 | 5.8 | 28.6 | 71.3 | 121.6 | 135.4 | 137.1 | 0.945 |

Experiment 2 for Dry Centrifugal Chromatography Cleaning Detection

Colloidal gold micro particle was used as a liquid phase reaction carrier, and method A was used to perform the experiment.

1. Experimental Materials

Referring to Experiment 1.

2. Experimental Methods

Preparation of human myoglobin solutions: referring to Experiment 1.

Colloidal gold micro particle labeling: referring to Experiment 1.

Preparation of colloidal gold micro particle-labeled membrane: referring to Experiment 1.

Preparation of polyclonal antibody-imprinted membrane: referring to Experiment 1.

Method for assembling semi-finished product: referring to Experiment 1.

Test group:

The test strip was directly centrifuged at 2,000 rpm for 30 seconds for cleaning without previously adding with 80 μL, of 50 mM PBS (pH 7.4) containing 0.05% Tween-20 on the colloidal gold micro particle-labeled membrane. Other procedures were referred to Experiment 1.

Control group:

Procedures were referred to Experiment 1 with removing the step of adding 80 μL of 50 mM PBS (pH 7.4) containing 0.05% Tween-20 on the colloidal gold particle-labeled membrane followed by a standing to allow the liquid flow completely into the nitrocellulose membrane.

3. Experimental Results

In the present experiment, the colloidal gold micro particle was used as a liquid phase reaction carrier, and method A was used to perform the experiment. As a result, the average detection processing time of the test strip of the test group of the present invention was 3.6 minutes, while that of the control group (without centrifugation processing) was 39 minutes. The test data of the test group of the present invention showed a good correlation between concentration and chroma value with a correlation coefficient r of 0.990 when the upper detection limit was set to 150 ng/mL, and the linear detection range was 3-150 ng/mL. As for the control group, when the upper detection limit was set to 100 ng/mL, the correlation coefficient r was 0.992, which met the detection requirement of r>0.98, and the linear detection range was 3-100 ng/mL. The ratio of the test data obtained at 10 ng/mL to that obtained at 3 ng/mL of the test group of the present invention was 1.26, while that of the control group was only 1.21. The above differences were mainly resulted from an undesirable cleaning effect of the colloidal gold micro particle due to a non-specific binding to the solid phase membrane, thereby resulting in a high background. The experimental results indicated that the technique of the present invention can not only reduce the detection time, but also improve the linearity and accuracy of the detection when compared with the prior art. The experimental results were shown in Table 27.

TABLE 27
Experimental results (chroma value) of effect of
the dry cleaning on the immune chromatography colloidal
gold assay with centrifuge isolation

| | Concentration (ng/mL) | | | | | | | |
|---|---|---|---|---|---|---|---|---|
| | 3 | 10 | 30 | 70 | 100 | 150 | 200 | r value |
| Test group | 5.7 | 7.2 | 27.9 | 66.8 | 112.8 | 138.5 | 141.2 | 0.963 |
| Control group | 7.5 | 9.1 | 28.1 | 72.3 | 119.2 | 132.5 | 139.7 | 0.953 |

Experiment 3 for Colloidal Gold Immune Chromatography Assay with Centrifuge Isolation Using Method B

Colloidal gold micro particle was used as a liquid phase reaction carrier, and method B was used to perform the experiment.

1. Experimental Materials

NHS-activated biotin (SIGMA Corporation), avidin (SIGMA Corporation), and other materials were referred to Experiment 1.

In the present experiment, the biotin was used an intermediate A and the avidin was used as an intermediate B. Others were referred to Experiment 1.

2. Experimental Methods

Preparation of human myoglobin solutions: referring to Experiment 1.

Biotin labeling of anti-human myoglobin polyclonal antibody:

The anti-human myoglobin polyclonal antibody was dialyzed against 0.1 M sodium carbonate buffer (pH 9.5) overnight and adjusted to a final concentration of 2 mg/mL. 20 mg of NHS-activated biotin was dissolved in 1 mL of dimethylformamide to produce a biotin solution. 50 μL of the biotin solution was added to the dialyzed antibody solution. The reaction was reacted at room temperature for 4 hours. The resulting product was dialyzed against PBS overnight and preserved at −20° C.

Colloidal gold micro particle labeling: referring to experiment 1.

Preparation of colloidal gold micro particle-labeled adsorption membrane:

The colloidal gold micro particle-labeled antibody solution was diluted to an OD₅₃₀ of 30 with a colloidal buffer (1% BSA, 3% sucrose, 50 mM PBS, pH 7.4). The diluted antibody solution was added with biotin-labeled anti-human myoglobin polyclonal antibody at an amount of 10 μg/mL and mixed evenly. Other procedures were referred to Experiment 1.

Preparation of avidin-imprinted membrane:

The avidin solution was diluted to a concentration of 1 mg/mL with 50 mM phosphate buffer (pH 7.4). The imprinter was started and loaded with the diluted avidin solution. Other procedures were referred to Experiment 1.

Method for assembling semi-finished product:

The dehumidifier was started to reduce the humidity in the operating room to less than 25%. The water-absorbing paper membrane pad and the colloidal gold particle-labeled adsorption membrane were pasted respectively on each end of the avidin-imprinted membrane. Other procedures were referred to Experiment 1.

Test group: referring to Experiment 1.

Control group: referring to Experiment 1.

3. Experimental Results

In the present experiment, the colloidal gold micro particle was used as a liquid phase reaction carrier, and method A was used for the experiment. As a result, the average detection processing time of the test strip of the test group of the present invention was 3.6 minutes, while that of the control group (without centrifugation processing) was 37 minutes. The test data of the test group of the present invention showed a good correlation between concentration and chroma value with a correlation coefficient r of 0.989 when the upper detection limit was set to 150 ng/mL, and the linear detection range was 3-150 ng/mL. As for the control group, when the upper detection limit was set to 100 ng/mL, the correlation coefficient r was 0.991, which met the detection requirement of r>0.98, and linear detection range was 3-100 ng/mL. The ratio of the test data obtained at 10 ng/mL to that obtained at 3 ng/mL of the test group of the present invention was 2.23, while that of the control group was only 1.52. The above differences were mainly resulted from an undesirable cleaning effect of the colloidal gold micro particle due to a non-specific binding to the solid phase membrane, thereby resulting in a high background. The experimental results indicated that the technique of the present invention can not only reduce the detection time, but also improve the linearity and accuracy of the detection when compared with the prior art. The experimental results were shown in Table 28.

TABLE 28
Experimental results (chroma value) of immune chromatography
colloidal gold assay with centrifuge isolation using method B

| | Concentration (ng/mL) | | | | | | | |
|---|---|---|---|---|---|---|---|---|
| | 3 | 10 | 30 | 70 | 100 | 150 | 200 | r value |
| Test group | 5.3 | 11.8 | 32.1 | 78.9 | 112.8 | 137.6 | 139.2 | 0.957 |
| Control group | 7.1 | 10.8 | 29.2 | 76.9 | 129.1 | 133.5 | 141.8 | 0.940 |

Experiment 4 for effect of centrifugal speed on test results

Colloidal gold micro particle was used as a liquid phase reaction carrier, and method A was used for the experiment.

1. Experimental Materials

Referring to Experiment 1.

2. Experimental Methods

Control group was not required. Human myoglobin solution of the test group was sampled respectively at a centrifugal speed of 500, 1,000, 2,000, 3,000, 4,000and 5,000 rpm. The centrifugation was performed after a standing for 1 minute after being added with the samples. The cleaning was performed at a centrifugal speed of 2,000 rpm. Other procedures were referred to Experiment 1.

3. Experimental Results

In the present experiment, the colloidal gold micro particle was used as a liquid phase reaction carrier, and method A was used to perform the experiment. Effects of different centrifugal speeds on the test results were investigated. Human myoglobin samples of different concentrations were tested in the experiment, and the experimental results were shown in Table 29. As shown in Table 29, the detection accuracy was related to the centrifugal speed. The test results obtained with centrifugal speeds of 500, 1,000 and 2,000 rpm met the requirement. While the test results obtained with centrifugal speeds of 3,000, 4,000 and 5,000 rpm indicated a significant decrease in chroma value, which affected the detection sensitivity and accuracy. Such results demonstrated that an optimal speed for detecting myoglobin in the present invention should be below 2,000 rpm.

TABLE 29
Effect of centrifugal speed on the test results (chroma value)

| Centrifugal speed | Concentration (ng/mL) | | | | | | | |
|---|---|---|---|---|---|---|---|---|
| rpm | 3 | 10 | 30 | 70 | 100 | 150 | 200 | r value |
| 500 | 4.1 | 8.2 | 26.7 | 68.1 | 108.2 | 129.7 | 135 | 0.963 |
| 1000 | 2.3 | 7.7 | 29.1 | 70.2 | 111.2 | 129.3 | 136.8 | 0.960 |
| 2000 | 3.5 | 8.2 | 28.7 | 71.2 | 107.9 | 131.6 | 138.7 | 0.966 |
| 3000 | 2.1 | 3.2 | 15.2 | 55.6 | 81.4 | 93.2 | 109.1 | 0.970 |
| 4000 | 3.2 | 2.8 | 7.6 | 25.6 | 56.4 | 68.1 | 73.2 | 0.966 |
| 5000 | 3.1 | 4.2 | 4.9 | 21.3 | 48.2 | 67.2 | 71.4 | 0.973 |

Experiment 6: Comparative Experiment for Time-Associated Detection Stability Between the Present Invention and the Prior Art

Colloidal gold micro particle was used as a liquid phase reaction carrier, and method A was used to perform the experiment.

1. Experimental Materials

Referring to Experiment 1.

2. Experimental Methods

Preparation of human myoglobin solutions

A human myoglobin solution of a known concentration was diluted to a human myoglobin solution of 50 ng/mL with a sample dilution buffer (1% BSA, 100 mM glycine, 50 mM PBS, 150 mM NaCl, pH 7.4).

Colloidal gold micro particle labeling: referring to Experiment 1.

Preparation of colloidal gold micro particle-labeled membrane: referring to Experiment 1.

Preparation of polyclonal antibody-imprinted membrane: referring to Experiment 1.

Method for assembling semi-finished product: referring to Experiment 1.

Test group 1:

Procedures were referred to Experiment 1. The test strip was cleaned with a cleaning solution through centrifugation. The test strip was placed on the colloidal gold quantitative chromatography analyzer for a reading of the chroma value, and the reading was performed every 2 minutes and performed 5 times successively. The experiment was repeated 3 times, and the results were averaged.

Test group 2:

Procedures were referred to Experiment 1. The test strip was cleaned using a dry centrifugal cleaning (without a cleaning solution). The test strip was placed on the colloidal gold quantitative chromatography analyzer for a reading of the chroma value. The reading was performed every 2 minutes and performed 5 times successively. The experiment was repeated 3 times, and the results were averaged.

Control group:

Procedures were referred to Experiment 1. The test strip was added with the sample followed by a standing for 20 minutes. The test strip was then placed on the colloidal gold quantitative chromatography analyzer for a reading of the chroma value. The reading was performed every 2 minutes and performed 5 times successively. The experiment was repeated 3 times, and the results were averaged.

3. Experimental results

In the present experiment, the colloidal gold micro particle was used as a liquid phase reaction carrier, and method A was used to perform the experiment. The stability of the chroma value with the extension of the standing time after the completion of the cleaning was investigated, and the experimental results were shown in Table 31. The test data of the centrifugal cleaning with the cleaning solution and dry centrifugal cleaning of the present invention was substantially stable during the subsequent 8-minute continuous observation period, while the test result of the control group obtained using a technique of the prior art increased over time during the subsequent 8-minute continuous observation period. The results indicated that the technique of the present invention can improve the stability of detection.

TABLE 31
Results (chroma value) of the comparative experiment for time-associated
detection stability between the present invention and the prior art

| | Delay detection time (min) | | | | |
|---|---|---|---|---|---|
| | 0 | 2 | 4 | 6 | 8 |
| Test group 1 | 52.3 | 51.9 | 53.2 | 52.1 | 52.7 |
| Test group 2 | 56.3 | 55.4 | 54.9 | 56.1 | 56.4 |
| Control group | 59.2 | 62.4 | 67.9 | 69.1 | 69.8 |

### EXAMPLE 4

### Preparation of Centrifugal Separation and Detection Device

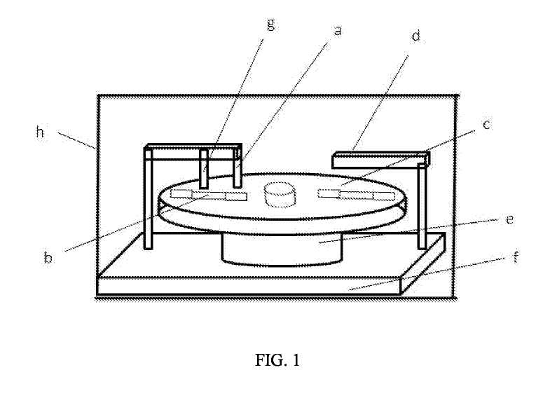

As shown in FIG. 1, the centrifugal separation and detection device of the present invention included: a sampling member a, a solid phase membrane b, a centrifugation device (a centrifugation rotor c; a drive motor e; a supporting base f) and a detector d, and an ultrasonication device k and an outer housing h. A proximal end of the solid phase membrane b corresponded to the sampling member a, and the ultrasonication device g corresponded to a detection portion of the solid phase membrane b. The centrifugation device consisted of the centrifugation rotor c, the drive motor e and the supporting base f. The drive motor e was arranged on the supporting base f, and the centrifugation rotor c was driven to rotate by the drive motor e.

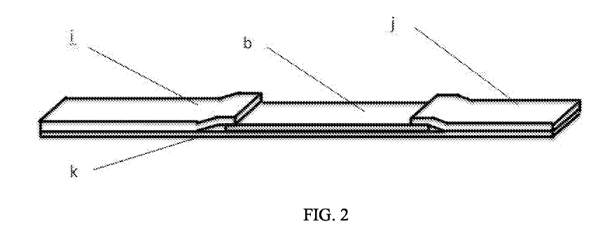

As shown in FIGS. 1 and 2, the solid phase membrane b was provided at a proximal end of the centrifugation rotor c with a liquid adsorption dispersion member i communicating therewith, and the solid phase membrane b is provided at a distal end of the centrifugal rotor c with a liquid collecting member h communicating therewith. The solid phase membrane b is fixedly arranged on an outer edge of the centrifugation rotor c. The solid phase membrane b and the centrifugation rotor c both had a detachable structure. The liquid absorption dispersion member i was used as a liquid phase sample loading portion of the sampling member. The material of the solid phase membrane b was any one of a nitrocellulose membrane, a polyvinylidene fluoride membrane, a nylon membrane and a DEAE cellulose membrane, and one or both sides of the solid phase membrane b were provided with a backing. The liquid adsorption dispersion member i included at least one of a colloidal gold-labeled adsorption membrane, a fluorescence-labeled antibody adsorption membrane, a chemiluminescence-labeled adsorption membrane and a dispersion membrane. A water-absorbing material, such as a water-absorbing paper and/or a water-absorbing gel, was used in the liquid collecting device h, and a liquid collecting container can also be used therein.

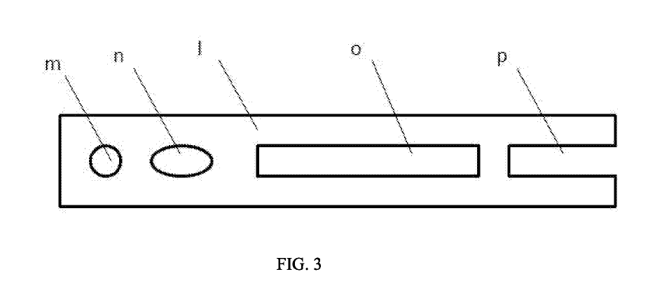

A fixer for fixing the solid phase membrane b was shown in FIGS. 2 and 3, which can be a support base plate k as shown in FIG. 2 and a lateral flow test strip buckle-like member 1 as shown in FIG. 3. The support base plate k can be selected from a PVC plate, a transparent plastic plate, an organic-glass plate, etc. The fixer shown in FIG. 3 was a lateral flow test strip buckle-like member 1, which specifically included a hole-shaped component m, a sampling groove n, an observation window o and a liquid collecting member outlet p. After the solid phase membrane structure was placed in the lateral flow test strip buckle-like structure 1, the sampling groove n was corresponded to the liquid adsorption dispersion member i, and the observation window o was corresponded to the solid phase membrane b, and the liquid collecting member outlet p was corresponded to the liquid collecting member h (a water-absorbing material or a liquid collecting container).

The following experiments are used to illustrate the methods of the present invention and effects thereof, but are not intended to limit the scope of the present invention. The experimental methods used in the experiments described below are conventional methods unless specified otherwise. Materials, reagents and the like used in the following experiments are commercially available unless specified otherwise.

Experiment 1. Preparation of the planar centrifugal separation and detection device of Example 1 and detection effect thereof. 1. Preparation of the device 1. 1 Experimental materials

Digital-display low speed centrifuge (rated output speed: 4,000 rpm, power: 250 w, planar centrifugation rotor, produced by Changzhou Yichen Instrument Company), Chuangrui injection pump (Baoding Chuangrui Pump Company, type Z S100-16/CR01), ultrasonicator (Shanghai Zhixin Instrument Company, type JDY-250), paper-chromatography detection card (Changzhou Biowin Company), human myoglobin (Sigma-Aldrich Corporation), colloidal gold quantitative chromatography analyzer (Skannex Corporation, Norway) , anti-human myoglobin polyclonal antibody (Genagates Corporation, USA), anti-human myoglobin monoclonal antibody (Genagates Corporation, USA), horseradish peroxidase (HRP, SIGMA Corporation), spectrophotometer (752 UV-Vis spectrophotometer, Shanghai Qinghua Technology Instruments Co., Ltd.), BioFlow imprinter (IMAGENE Corporation, USA), Index slitter (A-point Company, USA), DBF-900 sealing machine (Wenzhou Jiangnan Packaging Factory), ACBO dehumidifier (Jiangsu Wuxi Aobo Dehumidifier Company), desk centrifuge (Eppendorf Corporation, USA), bovine serum albumin (BSA, SIGMA Corporation), nitrocellulose membrane (MILLIPORE Corporation), polyester cellulose membrane (Alstrom Corporation, USA), water-absorbing paper membrane pad (S&S Company, USA), chloroauric acid (SIGMA Corporation), chemiluminescence detector (Glomax Multi JR Detection System, Promega Corporation), and West Pico luminescent reagent (Thermo Scientific Corporation).

In the present experiment, the digital-display low speed centrifuge was a planar centrifugal device; the Chuangrui injection pump was used as a sampling pump in a sampling member; the ultrasonicator was used as an ultrasonication device; the paper-chromatography detection card was a fixer; the colloidal gold quantitative chromatography analyzer was used as a detector (colloidal gold); the nitrocellulose membrane was used as a solid phase membrane; the polyester cellulose membrane was used as a liquid absorption dispersion member; the water-absorbing paper membrane pad was used as a liquid collecting member; and the chemiluminescence detector was used as a detector (chemiluminescence).

1.2 Experimental methods

1) Preparation of the planar centrifugal separation and detection device

The centrifugation rotor of the customized digital-display low speed centrifuge had a planar structure. A buckling groove for 16 paper-chromatography detection cards was arranged on a plane of the centrifugation rotor. The centrifugation rotor, below an observation window of the paper-chromatography detection card corresponding to the buckling groove, was provided with a vertically-through window. A drive motor and a supporting base were positioned below the centrifugation rotor. 16 Chuangrui injection pumps were correspondingly and fixedly mounted above the centrifugation rotor, and fixed to the supporting base which was below the injection pump. A fixing bracket of the ultrasonicator was prepared, and the ultrasonicator was fixed to the fixing bracket in a manner that an amplitude-transforming pole was corresponded to the vertically-through window arranged at the centrifugation rotor, and the amplitude-transforming pole can be moved up and down for an adjustment. A lower layer of the observation window of the paper-chromatography detection card was opened, thereby giving the observation window a vertically-through window structure. A myoglobin test strip (described below) was placed on the paper-chromatography detection card in such a manner that its water-absorbing membrane pad (i.e. a liquid collecting member) faced outward and its colloidal gold-labeled absorption membrane (i.e. a liquid absorption dispersion member) faced inwards, and was fixated by buckling an upper lid to make a myoglobin detection card. The myoglobin detection card was placed and fixed in the buckling groove of the centrifugation device. A chemiluminescence detector was placed on one side of the centrifugation detection device. Via the above procedures, the planar centrifugal separation and detection device was prepared.

2) Test for detection effect:

(1) Preparation of human myoglobin solutions: referring to Experiment 1 of Example 1.

(2) Colloidal gold micro particle labeling: referring to Experiment 1 of Example 1.

(3) Preparation of colloidal gold micro particle-labeled adsorption membrane: referring to Experiment 1 of Example 1.

(4) Preparation of polyclonal antibody-imprinted membrane: referring to Experiment 1 of Example 1.

(5) Method for assembling semi-finished product: referring to Experiment 1 of Example 1.

(6) Test group

The test strip prepared above was fixed in the paper-chromatography detection card with the lower layer of its observation window opened. The detection card with the test strip in was then placed in the centrifugation rotor with its colloidal gold particle-labeled absorption membrane as a proximal side. Then the Chuangrui injection pump was started. Other procedures were referred to Experiment 1 of Example 1.

Control group

The detection card was not subjected to a centrifugation and was placed on a table and manually loaded with the sample. The detection card was subjected to a standing each time after the sample was loaded to allow the liquid flow completely into the nitrocellulose membrane. Other procedures were referred to the test group.

1.3 Experimental results

The centrifugal separation and detection device of the present invention was compared to the existing conventional method, and the influence of the techniques of the present invention and the prior art on the detection result was investigated. According to the results, the average processing time for single detection of the test strip of the test group of the present invention was 4.5 minutes, while that of the control group (without centrifugation processing) was 47 minutes. The test data of the test group of the present invention showed a good correlation between concentration and luminescence value with a correlation coefficient r of 0.976. While the test data of the control group indicated substantially no correlation between concentration and luminescence value at a concentration below 100 ng/mL, and the correlation coefficient r was 0.821, P<0.05. The above differences were mainly resulted from an undesirable cleaning effect of the chemiluminescent enzyme due to a non-specific binding to the solid phase membrane, thereby resulting in a high background. The present invention has significantly better test results than those obtained using the assay without centrifugation processing, which indicated that the technique of the present invention can improve the linearity and accuracy of the detection when compared to the prior art. The experimental results were shown in Table. 34.

TABLE 34
Experimental results (luminescence value) of paper chromatography and
chemiluminescence assay using the device of the present invention

| | Concentration (ng/mL) | | | | | |
|---|---|---|---|---|---|---|
| | 0.1 | 1.0 | 10 | 100 | 1000 | r value |
| Test group | 15931 | 57643 | 193520 | 1252123 | 31862731 | 0.976 |
| Control group | 1021058 | 1267342 | 1198672 | 1359286 | 3457823 | 0.821 |

### INDUSTRIAL APPLICATION

The above technical solutions of the present invention have the following advantages.

1. The centrifugation device drives the detection liquid phase to flow on the solid phase membrane, which can effectively reduce the non-specific binding of the specifically captured chemiluminescent substance to the solid phase membrane, and reduce background noise interference from the solid phase membrane, and improve the detection sensitivity. In addition, a uniform flow of liquid on the membrane and a shorter detection time are also achieved. 2. The present invention uses a micro particle as a carrier of an analyte and an intermediate reaction substance thereof (or a colloidal gold micro particle) to chromatography and flow on the solid membrane to perform the reaction, which improves the ability of the solid phase membrane to capture and bind the nanlyte and the intermediate reaction substance thereof.

3. The existing flat-plate and magnetic-particle separation chemiluminescence detection techniques commonly adopt multi-step or multi-stage drive control, which involves transposition and movement of detection sample, detection phase and reaction carrier, and a temperature control. The present invention adopts a paper-chromatography method for chemiluminescence detection, which optimizes the reaction to a single mode at room temperature and in a chromatographic flow, thereby shortening the detection time, simplifying the operational procedures and the design, and reducing the cost of the instruments.

4. The existing paper-chromatography and fluorescence immunoassay technique adopts lateral flow control. The fluorescence intensity of the detection line is not only proportional to the concentration of the analyte, but also is positively correlated with the reaction time within a certain time range, which produces great difficulty in practical detection and control and an increase in detection error. The present invention adopts paper-chromatography centrifugation technique to perform fluorescence detection, which can significantly reduce the influence of detection time on detection result and improve detection accuracy.

5. The existing paper-chromatography colloidal gold immunoassay technique adopts lateral flow control. The color density of colloidal gold micro particles of the detection line is not only proportional to the concentration of the analyte, but also is positively correlated with the reaction time within a certain time range, which produces great difficulty in practical detection and control, and an increase in detection error. The invention adopts paper-chromatography centrifugation technique to detect the color density of colloidal gold micro particles, which can significantly reduce the influence of detection time on detection result and improve detection accuracy.

6. The invention uses a centrifugation device to drive the flow of detection liquid phase on the solid phase membrane and perform a cleaning step, which improves the ability to capture and bind to the analyte, reduces background noise interference from the solid phase membrane and improves detection sensitivity, thereby achieving a high-sensitivity detection with the existing detection reagents.

7. The invention uses a centrifugation device to drive the flow of detection liquid phase on the solid phase membrane which solves the existing problems that membrane detection technique is only performed using a natural flow and the liquid is reduced with the extension of the flow distance on the membrane. The present invention involves a constant flow of the liquid on the membrane, thus ensuring a binding uniformity of the analyte to the membrane and improving detection accuracy.

What is claimed is:

1. A combined kit of immune chromatography chemiluminescence assay, comprising:
an analyte-specific binding substance,
a micro particle,
a solid phase membrane,
a chemiluminescent reactant,
a centrifugation device, and
a luminescence detector;
wherein the chemiluminescent reactant comprises at least one of a chemiluminescent enzyme, a chemiluminescent substance, a chemiluminescent enzyme substrate and a luminescence-activating reagent;
the micro particle is a particle that is able to form a stable non-specific binding to a protein and/or the chemiluminescent reactant directly and/or by chemical crosslinking;
the solid phase membrane is a membranous substance forming a non-specific binding to a protein;
the centrifugation device centrifugally drives a liquid phase to flow on the solid phase membrane through chromatography;
the analyte-specific binding substance is selected from at least one of substances having a specific binding ability comprising an antigen, an antibody, an avidin and a biotin;
the chemiluminescent enzyme comprises at least one of horseradish peroxidase, alkaline phosphatase and xanthine oxidase;
the solid phase membrane is selected from any one of a nitrocellulose membrane, a polyvinylidene fluoride membrane, a nylon membraneand a DEAE cellulose membrane; and
the luminescence detector is a chemiluminescence detector.

2. The combined kit of claim 1, wherein the combined kit is used according to any one of the following methods A to F; wherein:
method A comprises:
(1) labeling the micro particle simultaneously with the analyte-specific binding substance and the chemiluminescent enzyme;
(2) reacting an analyte-containingsample with the labeled micro particle to achieve a binding between the analyte and the analyte-specific binding substance on the labeled micro particle, and forming a chemiluminescent enzyme-micro particle-analyte-specific binding substance-analyte complex (complex 1);
(3) coating a second analyte-specific binding substance which is capable of forming specific binding to the analyte on the complex 1 on the solid phase membrane;
(4) chromatographing a liquid phase containing the complex 1 to pass through the second analyte-specific binding substance coated on the solid phase membrane, and forming a chemiluminescent enzyme-micro particle-analyte-specific binding substance-analyte-the second analyte-specific binding substance complex (complex 2), and then capturing and immobilizing the complex 2 onto the solid phase membrane;
(5) cleaning the solid phase membrane to remove the unbound complex 1 and the remaining chemiluminescent enzyme thereon with a cleaning solution using centrifugal chromatography; and
(6) transferring the cleaned solid phase membrane in a solution of the chemiluminescent enzyme substrate for a reaction, and using the luminescence detector to detect a luminescence value generated from the chemiluminescent enzyme substrate through a reaction catalyzed by the chemiluminescent enzyme indirectly immobilized on the solid phase membrane, thereby determining a content of the analyte;
method B comprises:
(1) labeling the micro particle with the analyte-specific binding substance;
(2) labeling a specific conjugate of the analyte-specific binding substance with the chemiluminescent enzyme to form a chemiluminescent enzyme-specific conjugate of the analyte-specific binding substance (a chemiluminescent enzyme marker);
(3) reacting an analyte-containing sample with the labeled micro particle to form a binding between the analyte and the analyte-specific binding substance on the labeled micro particle, thereby forming a micro particle-analyte-specific binding substance-analyte complex (complex 3);
(4) coating the second analyte-specific binding substance on the solid phase membrane;
(5) chromatographing a liquid phase containing the complex 3 to pass through the second analyte-specific binding substance coated on the solid phase membrane, and forming a micro particle-analyte-specific binding substance-analyte-the second analyte-specific binding substance complex (complex 4), and then capturing and immobilizing the complex 4 onto the solid phase membrane;
(6) chromatographing a liquid phase containing the chemiluminescent enzyme marker to pass through the complex 4 captured on the solid phase membrane, and forming a chemiluminescent enzyme marker-complex 4 complex (complex 5), through a binding between the chemiluminescent enzyme marker and the analyte-specific binding substance, and then capturing and immobilizing the complex 5 onto the solid phase membrane;
(7) cleaning the solid phase membrane to remove the unbound chemiluminescent enzyme marker and the remaining chemiluminescent enzymethereon with a cleaning solution using centrifugal chromatography; and
(8) transferring the cleaned solid phase membrane in a solution of the chemiluminescent enzyme substrate for a reaction, and using the luminescence detector to detect a luminescence value generated from the chemiluminescent enzyme substrate through a reaction catalyzed by the chemiluminescent enzyme indirectly immobilized on the solid phase membrane, thereby determining a content of the analyte;
method C comprises:
(1) labeling the micro particle simultaneously with the analyte-specific binding substance and the chemiluminescent enzyme;
(2) reacting an analyte-containing sample with the labeled micro particle to achieve a binding between the analyte and the analyte-specific binding substance on the labeled micro particle, and forming a chemiluminescent enzyme-micro particle-analyte-specific binding substance-analyte complex (complex 1);
(3) labeling the second analyte-specific binding substance with an intermediate A;
(4) coating an intermediate B capable of forming a specific binding to the intermediate A on the solid phase membrane;
(5) reacting a liquid phase containing the complex 1 with the second analyte-specific binding substance labeled with the intermediate A to form a chemiluminescent enzyme-micro particle-analyte-specific binding substance-analyte-second analyte-specific binding substance-intermediate A complex (complex 6);
(6) chromatographing a liquid phase containing the complex 6 to pass through the intermediate B coated on the solid phase membrane, and forming a chemiluminescent enzyme-micro particle-analyte-specific binding substance-analyte-second analyte-specific binding substance-intermediate A-intermediate B complex (complex 7), and then capturing and immobilizing the complex 7 on the solid phase membrane;
(7) cleaning the solid phase membrane to remove the unbound complex 6 and the remaining chemiluminescent enzyme thereon with a cleaning solution using centrifugal chromatography; and
(8) transferring the cleaned solid phase membrane in a solution of the chemiluminescent enzyme substrate for a reaction, and using the luminescence detector to detect a luminescence value generated from the chemiluminescent enzyme substrate through a reaction catalyzed by the chemiluminescent enzyme indirectly immobilized on the solid phase membrane, thereby determining a content of the analyte;
method D comprises:
(1) labeling the micro particle with the analyte-specific binding substance;
(2) labeling a specific conjugate of the analyte-specific binding substance with the chemiluminescent enzyme to form a chemiluminescent enzyme-specific conjugate of the analyte-specific binding substance (a chemiluminescent enzyme marker);
(3) reacting an analyte-containing sample with the labeled micro particle to form a binding between the analyte and the analyte-specific binding substance on the labeled micro particle, thereby forming a micro particle-analyte-specific binding substance-analyte complex (complex 3);
(4) labeling the second analyte-specific binding substance with the intermediate A;
(5) coating the intermediate B capable of forming a specific binding to the intermediate A on the solid phase membrane;
(6) reacting a liquid phase containing the complex 3 with the second analyte-specific binding substance labeled with the intermediate A to form a micro particle-analyte-specific binding substance-analyte-second analyte-specific binding substance-intermediate A complex (complex 8);
(7) chromatographing a liquid phase containing the complex 8 to pass through the intermediate B coated on the solid phase membrane, and forming a micro particle-analyte-specific binding substance-analyte-second analyte-specific binding substance-intermediate A-intermediate B complex (complex 9), and then capturing and immobilizing the complex 9 on the solid phase membrane;
(8) chromatographing a liquid phase containing the chemiluminescent enzyme marker to pass through the complex 9 captured on the solid phase membrane, and forming a chemiluminescent enzyme-specific conjugate of analyte-specific binding substance-complex 9 complex through a binding with the analyte-specific binding substance (complex 10), and then capturing and immobilizing the complex 10 on the solid phase membrane;
(9) cleaning the solid phase membrane to remove the unbound chemiluminescent enzyme marker and the remaining chemiluminescent enzymethereon with a cleaning solution using centrifugal chromatography; and
(10) transferring the cleaned solid phase membrane in a solution of the chemiluminescent enzyme substrate for a reaction, and using the luminescence detector to detect a luminescence value generated from the chemiluminescent enzyme substrate through a reaction catalyzed by thechemiluminescent enzyme indirectly immobilized on the solid phase membrane, thereby determining a content of the analyte;
method E comprises:
(1) labeling the micro particle with the analyte-specific binding substance and the chemiluminescent substance;
(2) reacting an analyte-containing sample with the labeled micro particle to form a chemiluminescent substance-micro particle-analyte-specific binding substance-analyte complex (complex 11), through a binding between the analyte and the analyte-specific binding substance on the labeled micro particle;
(3) coating the second analyte-specific binding substance on the solid phase membrane;
(4) chromatographing a solid phase containing the complex 11 to pass through the second analyte-specific binding substance coated on the solid phase membrane, and forming a chemiluminescent substance-micro particle-analyte-specific binding substance-analyte-second analyte-specific binding substance complex (complex 12), and capturing and immobilizing the complex 12 on the solid phase membrane;
(5) cleaning the solid phase membrane to remove the unbound complex 11 and the remaining chemiluminescent substance with a cleaning solution using centrifugal chromatography; and
(6) transferring the cleaned solid phase membrane in a solution of the luminescence-activating reagent for a reaction, and using the luminescence detector to detect a luminescence value generated from decomposition of the chemiluminescent substance indirectly immobilized on the solid phase membrane, thereby determining a content of the analyte;
method F comprises:
(1) labeling the micro particle with the analyte-specific binding substance;
(2) labeling a specific conjugate of the analyte-specific binding substance with the chemiluminescent substance to form a chemiluminescent substance-specific conjugate of the analyte-specific binding substance (a chemiluminescent substance marker);
(3) reacting an analyte-containing sample with the labeled micro particle to form the micro particle-analyte-specific binding substance-analyte complex (complex 3), through a binding between the analyte and the analyte-specific binding substance on the labeled micro particle;
(4) coating the second analyte-specific binding substance on the solid phase membrane;
(5) chromatographing a solid phase containing the complex 3 to pass through the second analyte-specific binding substance coated on the solid phase membrane, and forming the micro particle-analyte-specific binding substance-analyte-second analyte-specific binding substance complex (complex 4), and capturing and immobilizing the complex 4 on the solid phase membrane;
(6) chromatographing a solid phase containing the chemiluminescent substance marker to pass through the complex 4 captured on the solid phase membrane, and forming a chemiluminescent substance-specific conjugate of the analyte-specific binding substance-complex 4 complex (complex 13), and capturing and immobilizing the complex 13 on the solid phase membrane;
(7) cleaning the solid phase membrane to remove the unbound chemiluminescent substance marker and the remaining chemiluminescent substance with a cleaning solution using centrifugal chromatography; and
(8) transferring the cleaned solid phase membrane in a solution of the luminescence-activating reagent for a reaction, and using the luminescence detector to detect a luminescence value generated from decomposition of the chemiluminescent substance indirectly immobilized on the solid phase membrane, thereby determining a content of the analyte.

3. A combined kit of immune chromatography fluorescence assay with centrifuge isolation, comprising:
an analyte-specific binding substance,
a micro particle,
a solid phase membrane,
a fluorescent substance,
a centrifugation device,
and a fluorescence detector;
wherein the fluorescent substance comprises at least one of an organic fluorescent dye and a rare-earth element fluorescent dye;
the micro particle is able to form a stable non-specific binding to a protein and/or the fluorescent substance directly and/or by chemical crosslinking;
the solid phase membrane is a membranous substance with a non-specific binding characteristic to a protein;
the centrifugation device centrifugally drives a liquid phase to flow on the solid phase membrane through chromatography; and
the analyte-specific binding substances selected from at least one of substances having a specific binding ability comprising an antigen, an antibody, an avidin and a biotin.

4. The combined kit of claim 3, wherein the combined kit is used according to any one of the following methods A to D; wherein:
method A comprises:
(1) labeling the micro particle simultaneously with the analyte-specific binding substance and the fluorescent substance;
(2) reacting an analyte-containing sample with the labeled micro particle to form a fluorescent substance-micro particle-analyte-specific binding substance-analyte complex (complex 1′), through a binding between the analyte and the analyte-specific binding substance on the labeled micro particle;
(3) coating a second analyte-specific binding substance capable of forming a specific binding to the analyte on the complex 1′ on the solid phase membrane;
(4) chromatographing a liquid phase containing the complex 1′ to pass through the second analyte-specific binding substance coated on the solid phase membrane, and forming a fluorescent substance-micro particle-analyte-specific binding substance-analyte-second analyte-specific binding substance complex (complex 2′), and capturing and immobilizing the complex 2′ on the solid phase membrane;
(5) cleaning the solid phase membrane to remove the unbound complex 1′ and the remaining fluorescent substance with a cleaning solution using centrifugal chromatography; and
(6) detecting a luminescence value of the fluorescent substance immobilized on cleaned solid phase membrane generated by exciting light using a fluorescence detector to determine a content of the analyte;
method B comprises:
(1) labeling the micro particle with the analyte-specific binding substance;
(2) labeling a specific conjugate of the analyte-specific binding substance with the fluorescent substance to form a fluorescent substance-specific conjugate of the analyte-specific binding substance (a fluorescent substance marker);
(3) reacting an analyte-containing sample with the micro particle to form a micro particle-analyte-specific binding substance-analyte complex (complex 3′), through a binding between the analyte and the analyte-specific binding substance on the labeled micro particle;
(4) coating the second analyte-specific binding substance on the solid phase membrane;
(5) chromatographing a liquid phase containing the complex 3′ to pass through the second analyte-specific binding substance coated on the solid phase membrane, and forming a micro particle-analyte-specific binding substance-analyte-second analyte-specific binding substance complex (complex 4′), and capturing and immobilizing the complex 4′ on the solid phase membrane;
(6) chromatographing a liquid phase containing the fluorescent substance to pass through the complex 4′ captured on the solid phase membrane, and forming a fluorescent substance marker-complex 4′ complex (complex 5′), through a binding with the analyte-specific binding substance on the complex 4′, and capturing and immobilizing the complex 5′ on the solid phase membrane;
(7) cleaning the solid phase membrane to remove the unbound fluorescent substance marker and the remaining fluorescent substance with a cleaning solution using centrifugal chromatography; and
(8) detecting a luminescence value of the fluorescent substance immobilized on cleaned solid phase membrane generated by exciting light using a fluorescence detector to determine a content of the analyte;
method C comprises:
(1) labeling the micro particle simultaneously with the analyte-specific binding substance and the fluorescent substance;
(2) reacting an analyte-containing sample with the labeled micro particle to form thefluorescent substance-micro particle-analyte-specific binding substance-analyte complex (complex 1′), through a binding between the analyte and the analyte-specific binding substance on the labeled micro particle;
(3) labeling the second analyte-specific binding substance with an intermediate A;
(4) coating an intermediate B capable of forming a specific binding to the intermediate A on the solid phase membrane;
(5) reacting a liquid phase containing the complex 1′ with the second analyte-specific binding substance labeled with the intermediate A to form a fluorescent substance-micro particle-analyte-specific binding substance-analyte-second analyte-specific binding substance-intermediate A complex (complex 6′);
(6) chromatographing a liquid phase containing the complex 6′ to pass through the intermediate B coated on the solid phase membrane, and forming a fluorescent substance-micro particle-analyte-specific binding substance-analyte-second analyte-specific binding substance-intermediate A-intermediate B complex (complex 7′), and capturing and immobilizing the complex 7′ on the solid phase membrane;
(7) cleaning the solid phase membrane to remove the unbound complex 6′ and the remaining fluorescent substance with a cleaning solution using centrifugal chromatography; and
(8) detecting a luminescence value of the fluorescent substance immobilized on cleaned solid phase membrane generated by exciting light using a fluorescence detector to determine a content of the analyte;
method D comprises:
(1) labeling the micro particle with the analyte-specific binding substance;
(2) labeling the specific conjugate of the analyte-specific binding substance with the fluorescent substance to form the fluorescent substance-specific conjugate of the analyte-specific binding substance complex (a fluorescent substance marker);
(3) reacting an analyte-containing sample with the labeled micro particle to form the micro particle-analyte-specific binding substance-analyte complex (complex 3′), through a binding between the analyte and the annlyte-specific conjugate on the labeled micro particle;
(4) labeling the second analyte-specific binding substance with the intermediate A;
(5) coating the intermediate B capable of forming a specific binding to the intermediate A on the solid phase membrane;
(6) reacting a liquid phase containing the complex 3′ with the second analyte-specific binding substance labeled with the intermediate A to form a micro particle-analyte-specific binding substance-analyte-second analyte-specific binding substance-intermediate A complex (complex 8′);
(7) chromatographing a liquid phase containing the complex 8′ to pass through the intermediate B coated on the solid phase membrane, and forming a micro particle-analyte-specific binding substance-analyte-second analyte-specific binding substance-intermediate A-intermediate B complex (complex 9′), and capturing and immobilizing the complex 9′ on the solid phase membrane;
(8) chromatographing a liquid phase containing the fluorescent substance marker to pass through the complex 9′ captured on the solid phase membrane, and forming a fluorescent substance-specific conjugate of the analyte-specific binding substance-complex 9′ complex (complex 10′), through a binding with the analyte-specific binding substance, and capturing and immobilizing the complex 10′ on the solid phase membrane;
(9) cleaning the solid phase membrane to remove the unbound fluorescent substance marker and the remaining fluorescent substance with a cleaning solution using centrifugal chromatography; and
(10) detecting a luminescence value of the fluorescent substance immobilized on cleaned solid phase membrane generated by exciting light using a fluorescence detector to determine a content of the analyte.

5. A combined kit of immune chromatography colloidal gold assay with centrifuge isolation, comprising an analyte-specific binding substance, a colloidal gold micro particle, a solid phase membrane, a centrifugal device and a colloidal gold detector;
wherein the colloidal gold micro particle is prepared using a gold solution and forms a stable non-specific binding to a protein directly;
the solid phase membrane is membranous substance with a non-specific binding characteristic to a protein;
the centrifugal device centrifugally drives a liquid phase to flow on the solid phase membrane through chromatography;
the analyte-specific binding substance is selected from at least one of substances having a specific binding ability comprising an antigen, an antibody, an avidin and a biotin; and
the colloidal gold detector is used to perform a quantitative or semi-quantitative detection through colorimetric analysis of a colloidal gold color.

6. The combined kit of claim 5, wherein the combined kit is used according to any one of the following methods A and B; wherein:
method A comprises:
(1) preparing the colloidal gold micro particle with a gold solution;
(2) labeling the colloidal gold micro particle with the analyte-specific binding substance;
(3) reacting an analyte-containing sample with the labeled colloidal gold micro particle to form a colloidal gold micro particle-analyte-specific binding substance-analyte complex (complex 1″), through a binding between the analyte and the analyte-specific binding substance on the labeled colloidal gold micro particle;
(4) coating a second analyte-specific binding substance capable of forming a specific binding to the analyte on the complex 1″ on the solid phase membrane;
(5) chromatographing a liquid phase containing the complex 1″ to pass through the second analyte-specific binding substance coated on the solid phase membrane, and forming a colloidal gold micro particle-analyte-specific binding substance-analyte-second analyte-specific binding substance complex (complex 2″), and capturing and immobilizing the complex 2″ on the solid phase membrane;
(6) cleaning the solid phase membrane to remove the unbound complex 1″ and the remaining colloidal gold with a cleaning solution using centrifugal chromatography; and
(7) detecting a luminescence value of the fluorescent substance immobilized on cleaned solid phase membrane generated by exciting light using a fluorescence detector to determine a content of the analyte;
method B comprises:
(1) preparing the colloidal gold micro particle with a gold solution;
(2) labeling the colloidal gold micro particle with the analyte-specific binding substance;
(3) reacting an analyte-containing sample with the labeled colloidal gold micro particle to form the colloidal gold micro particle-analyte-specific binding substance-analyte complex (complex 1″), through a binding between the analyte and the analyte-specific binding substance on the labeled colloidal gold micro particle;
(4) labeling the second analyte-specific binding substance with an intermediate A;
(5) coating an intermediate B capable of forming a specific binding to the intermediate A on the solid phase membrane;
(6) reacting a liquid phase containing the complex 1″ with the second analyte-specific binding substance labeled with the intermediate A to form a colloidal gold micro particle-analyte-specific binding substance-analyte-second analyte-specific binding substance-intermediate A complex (complex 3″);
(7) chromatographing a liquid phase containing the complex 3″ to pass through the intermediate B coated on the solid phase membrane, and forming a colloidal gold micro particle-analyte-specific binding substance-analyte-second analyte-specific binding substance-intermediate A-intermediate B complex (complex 4″), and capturing and immobilizing the complex 4″ on the solid phase membrane;
(8) cleaning the solid phase membrane to remove the unbound complex 3″ and the remaining colloidal gold with a cleaning solution using centrifugal chromatography; and
(9) detecting a luminescence value of the fluorescent substance immobilized on cleaned solid phase membrane generated by exciting light using a fluorescence detector to determine a content of the analyte.

7. A centrifugal separation and detection device, comprising:
a sampling member,
a solid phase membrane,
a centrifugal device,
and a detector;
wherein the centrifugal device comprises a centrifugal rotor driven by a drive motor and a support base, and the centrifugal rotor is supported by the support base;
the sampling member is not directly connected to the centrifugal rotor, and is arranged above, below or outside the centrifugal rotor;
the sampling member comprises a liquid phase storage device, an injection tube and an injection pump; and the liquid phase storage device is communicated with the injection tube; and a liquid in the liquid phase storage device is driven to enter into the injection tube with the injection pump;
the solid phase membrane is arranged on the centrifugal rotor and a liquid phase sample is loaded directly or indirectly to a proximal side of the solid phase membrane with the injection tube; and
the detector is not connected directly to the centrifugal rotor and is arranged above, below or outside the centrifugal rotor.
